SEQUENCE LISTING (10 sequences)

SEQ ID NO: 1 - DNA, 28038 bp, PEDV
acttaaagagattttctatctacggatagttagctctttttctagactcttgtctactcaattcaactaaacgaaattttgtccttccggccgcatgtccatgctgctggaagctgacgtggaatttcattaggtttgcttaagtagccatcgcaagtgctgtgctgtcctctagttcctggttggcgttccgtcgccttctacatactagacaaacagccttcctccggttccgtctgggggttgtgtggataactagttctgtctagtttgaaaccagtaactgtcggctatggctagcaaccatgttacattggcttttgccaatgatgcagaaatttcagcttttggcttttgcactgctagtgaagccgtctcatactattctgaggccgccgctagtggatttatgcaatgccgtttcgtgtccttcgatctcgctgacactgttgagggattgcttcccgaagactatgtcatggtggtggtcggcactaccaagcttagtgcgtatgtggacacttttggtagccgccccaaaaacatttgtggttggctgttattttctaactgtaattacttcctcgaagagttagagcttacttttggtcgtcgtggtggtaacatcgtgccagttgaccaatacatgtgtggcgctgacggtaaacctgttcttcaggaatccgaatgggagtatacagatttctttgctgactccgaagacggtcaactcaacattgctggtatcacttatgtgaaggcctggattgtagagcgatcggatgtctcttatgcgagtcagaatttaacatctattaagtctattacttactgttcaacctatgagcatacttttcctgatggtactgccatgaaggttgcacgtactccaaagattaagaagactgttgtcttgtctgagccacttgctactatctacagggaaattggttctccttttgtggataatgggagcgatgctcgttctatcattaagagaccagtgttcctccacgcttttgttaagtgtaagtgtggtagttatcattggactgttggtgattggacttcctatgtctccacttgctgtggctttaagtgtaagccagtccttgtggcttcatgctctgctacgcctggttctgttgtggttacgcgcgctggtgctggcactggtgttaagtattacaacaacatgttcctgcgccatgtggcagacattgatgggttggcattctggcgaattctcaaggtgcagtccaaagacgacctcgcttgctctggtaaattccttgaacaccatgaggaaggtttcacagatccttgctactttttgaatgactcgagcattgctactaagctcaagtttgacatccttagtggcaagttttctgatgaagtcaaacaagctatctttgctggtcatgttgttgttggcagcgcgctcgttgacattgttgacgatgcactgggacagccttggtttatacgtaagcttggtgaccttgcaagtgcagcttgggagcagcttaaggctgtcgttagaggccttaacctcctgtctgatgaggtcgtgctctttggcaaaagacttagctgtgccactcttagtatcgttaacggtgtttttgagttcatcgccgaagtgcctgagaagttggctgcggctgttacagtttttgtcaacttcttgaatgagctttttgagtctgcctgtgactgcttaaaggtcggaggtaaaacctttaacaaggttggctcttatgttctttttgacaacgcattggttaagcttgtcaaggcaaaagttcgcggcccacgacaggcaggtgtttgtgaagttcgttacacaagccttgttattgggagtactaccaaggtggtttccaagcgcgttgaaaatgccaatgtgaatctcgtcgtcgttgacgaggatgtgaccctcaacaccactggtcgtacagttgttgttgacggacttgcattcttcgagagtgacgggttttacagacatcttgctgatgctgacgttgtcattgaacatcctgtttataagtctgcttgtgagctcaagccagtttttgagtgtgacccaatacctgattttcctatgcctgtggccgctagtgttgcagagctttgtgtgcaaactgatctgttgcttaaaaattacaacactccttataaaacttacagctgcgttgtgagaggtgataagtgttgtatcacttgcaccttacatttcacagcaccaagttatatggaggctgctgctaattttgtagacctctgtaccaagaacattggtactgctggttttcatgagttttacattacggcccatgaacaacaggatctgcaagggttcgtaaccacttgttgcacgatgtcaggttttgagtgttttatgcctataatcccacagtgtccagcagtgcttgaagagattgatggtggtagcatctggcggtcttttatcactggtcttaatacaatgtgggatttttgcaagcatcttaaagtcagctttggactagatggcattgttgtcactgtagcacgcaaatttaaacgacttggtgctctcttggcagaaatgtataaaacttacctttcaactgtggtggaaaacttggtactggccggtgttagcttcaagtattatgccaccagtgtcccaaaaattgttttgggctgttgttttcacagtgttaaaagtgttcttgcaagtgccttccagattcctgtccaggcaggcgttgagaagtttaaagtcttccttaactgtgttcaccctgttgtaccacgtgtcattgaaacttcttttgtggaattagaagagacgacatttaaaccaccagcactcaatggtagtattgctattgttgatggctttgctttctattatgatggaacactatactatcccaccgatggtaatagcgttgttcctatctgctttaagaagaaaggtggtggtgatgtcaaattctctgatgaagtctctgttaaaaccattgacccagtttataaggtctcccttgaatttgagttcgagtctgagactattatggctgtgcttaataaggctgttggtaattgtatcaaggttacaggtggttgggacgatgttgttgagtatatcaatgttgccattgaggttcttaaagatcacatcgatgtgcctaagtactacatctatgatgaggaaggtggcaccgatcctaatctgcccgtaatggtttctcagtggccgttgaatgatgacacgatctcacaggatctgcttgatgttgaagttgttactgatgcgccagttgatttcgagggtgatgaagtagactcctctgaccctgataaggtggcagacgtggctaactctgagcctgaggatgacggtcttaatgtagctcctgaaacaaatgtagagtctgaagttgaggaagttgccgcaaccttgtcctttattaaagatacaccttccacagttactaaggatccttttgcttttgactttgcaagctatggaggacttaaggttttaagacaatctcataacaactgctgggttacttctaccttggtgcagctacaattgcttggcatcgttgatgaccctgcaatggagctttttagtgctggtagagttggtccaatggttcgcaaatgctatgagtcacaaaaggctatcttgggatctttgggtgatgtgtcggcttgcctagagtctctgactaaggacctacacacacttaagattacctgttctgtagtctgtggttgtggtactggtgaacgtatctatgatggttgtgcttttcgtatgacgccaactttggaaccgttcccatatggtgcttgtgctcagtgtgctcaagttttgatgcacacttttaaaagtattgttggcaccggcatcttttgtcgagatactactgctctctccttggattctttggttgtaaaacctctttgtgcggctgcttttataggcaaggatagtggtcattatgtcactaacttttatgatgctgctatggctattgatggttatggtcgtcatcagataaagtatgacacactgaacactatttgtgttaaagacgttaattggacagcaccttttgtcccagacgttgagcctgtattggagcttgttgtcaaacctttctattcttataagaatgttgatttttaccaaggagattttagtgaccttgttaaacttccatgtgattttgttgttaatgctgcaaatgagaatttgtctcacggtggcggcatagcaaaggccattgatgtttataccaagggcatgttgcagaagtgctcgaatgattacattaaagcacacggtcccattaaagttggacgtggtgtcatgttggaggcattaggtcttaaggtctttaatgttgttggtccacgtaagggtaagcatgcacctgagcttcttgttaaggcttataagtccgtttttgctaattcaggtgttgctcttacacctttgattagtgttggaatttttagtgttcctttggaagaatctttatctgcttttcttgcatgtgttggtgatcgccactgtaagtgcttttgttatagtgacaaagagcgcgaggcgatcattaattacatggatggcttggtagatgctattttcaaagatgcacttgttgatactactcctgtccaggaagatgttcaacaagtttcacaaaaaccagttttgcctaattttgaacctttcaggattgaaggtgctcatgctttctatgagtgcaaccctgaaggtttgatgtcattaggtgctgacaagctggtgttgtttacaaattccaatttggatttttgtagcgttggtaagtgtcttaacaatgtgactggcggtgcattgcttgaagccataaatgtatttaaaaagagtaacaaaacagtgcctgctggcaactgtgttacttttgagtgtgcagatatgatttctattactatggtagtattgccatctgacggtgatgctaattatgacaaaaattatgcacgcgccgtcgtcaaggtatctaagcttaaaggcaagttattgcttgctgttggtgatgccatgttgtattccaagttgtcccacctcagcgtgttaggtttcgtatccacacctgatgatgtggagcgtttctacgcaaataagagtgtggttattaaagttactgaggatacacgtagtgttaagactgttaaagtagaatccactgttacttatggacaacaaattggaccttgtcttgttaatgacaccgttgtcacagacaacaaacctgttgttgctgatgttgtagctaaggttgtaccaagtgctaattgggattcacattatggttttgataaggctggtgagttccacatgctagaccatactgggtttgcctttcctagtgaagttgttaacggtaggcgtgtgcttaaaaccacagataataactgttgggttaatgttacatgtttacaattacagtttgctagatttaggttcaagtcagcaggtctacaggctatgtgggagtcctattgtactggtgatgttgctatgtttgtgcattggttgtactggcttactggtgttgacaaaggtcagcctagtgattcagaaaatgcacttaacatgttgtctaagtacattgttcctgctggttctgtcactattgaacgtgtcacgcatgacggttgttgttgtagtaagcgtgttgtcactgcaccagttgtgaatgctagcgtgttgaagcttggcgtcgaggatggtctttgtccacatggtcttaactacattgacaaagttgttgtagttaaaggtactacaattgttgtcaatgttggaaaacctgtagtggcaccatcgcacctctttcttaagggtgtttcctacacaacattcctagataatggtaacggtgttgccggccattatactgtttttgatcatgacactggtatggtgcatgatggagatgtttttgtaccaggtgatctcaatgtgtctcctgttacaaatgttgtcgtctcagagcagacggctgttgtgattaaagaccctgtgaagaaagtagagttagacgctacaaagctgttagacactatgaattatgcatcggaaagattcttttcctttggtgattttatgtcacgtaatttaattacagtgtttttgtacatccttagtattttgggtctctgttttagggcctttcgtaagagggatgttaaagttctagctggtgtaccccaacgtactggtattatattgcgtaaaagtgtgcgctataatgcaaaggctttgggtgtcttcttcaagctaaaactttattggttcaaagttcttggtaagtttagtttgggtatttatgcattgtatgcattactattcatgacaatacgctttacacctataggtggccctgtttgtgatgatgttgttgctggttatgctaattctagttttgacaagaatgagtattgcaacagtgttatttgtaaggtctgtctctatgggtaccaggaactttcggacttctctcacacacaggtagtatggcaacaccttagagacccattaattggtaatgtgatgcctttcttttatttggcatttctggcaatttttgggggtgtttatgtaaaggctattactctctattttattttccagtatcttaacatacttggtgtgtttttgggcctacaacagtccatttggtttttgcagcttgtgccttttgatgtctttggtgacgagatcgtcgtctttttcatcgttacacgcgtattgatgttccttaagcatgttttccttggctgcgataaggcatcttgtgtggcttgctctaagagtgctcgccttaagcgcgttcctgtccagactatttttcagggtactagcaaatccttctacgtacatgccaatggtggttctaagttctgtaagaagcacaatttcttttgtttaaattgtgattcttatggtccaggctgcacttttattaatgacgtcattgcaactgaagttggtaatgttgtcaaacttaatgtgcaaccgacaggtcctgccactattcttattgacaaggttgaattcagtaatggtttttactatctttatagtggtgacacattttggaagtacaactttgacataacagataacaaatacacttgcaaagagtcacttaaaaattgtagcataatcacagactttattgtttttaacaataatggttccaatgtaaatcaggttaagaatgcatgtgtgtatttttcacagatgctttgtaaacctgttaagttagtggactcagcgttgttggccagtttgtctgttgattttggtgcaagcttacatagtgcttttgttagtgtgttgtcgaatagttttggcaaagacctgtcaagttgtaatgacatgcaggattgcaagagcacattgggttttgatgatgtaccattggatacctttaatgctgctgttgctgaggctcatcgttacgatgtcctcttgactgacatgtcgttcaacaattttaccaccagttatgcaaaaccagaggaaaaacttcccgtccatgacattgccacgtgtatgcgtgtaggtgccaagattgttaatcataacgttcttgtcaaggatagtatacctgtggtgtggcttgtacgtgatttcattgccctttctgaagaaactaggaagtacattattcgtacgactaaagttaagggtataaccttcatgttgacctttaatgattgtcgtatgcatactaccatacctactgtttgcattgcaaataagaagggtgcaggtcttcctagtttttcaaaggttaagaaattcttctggtttttgtgtctgttcatagttgctgttttctttgcactaagcttttttgattttagtactcaggttagcagtgatagtgattatgacttcaagtatattgagagtggccagttgaagacttttgacaatccacttagttgtgtgcataatgtctttagtaacttcgaccagtggcatgatgccaagtttggtttcacccccgtcaacaatcctagttgtcctatagtcgttggtgtatcagacgaagcgcgcactgttccaggtatcccagcaggtgtttatttagctggtaaaacacttgtttttgctattaacaccatttttggtacatctggtttgtgctttgatgctagtggcgttgctgataagggcgcttgcatttttaattcggcttgcaccacattatctggtttgggtggaactgctgtctactgttataagaatggtctagttgaaggtgctaaactttatagtgagttggcacctcatagctactataaaatggtagatggtaatgctgtgtctttacctgaaattatctcacgcggctttggcatccgtactatccgtacaaaggctatgacctactgtcgcgttggccagtgtgtgcaatctgcagaaggtgtttgttttggcgccgatagattctttgtctataatgcagaatctggttctgactttgtttgtggcacagggctctttacattgttgatgaacgttattagtgttttttccaagacagtaccagtaactgtgttgtctggtcaaatactttttaattgcattattgcttttgctgctgttgcggtgtgtttcttatttacaaagtttaagcgcatgttcggtgatatgtctgttggcgttttcactgtcggtgcttgtactttgttgaacaatgtttcctacattgtaacacagaacacacttggcatgttgggctatgcaactttgtactttttgtgcactaaaggtgttagatatatgtggatttggcatttgggatttttgatctcatatatacttattgcaccatggtgggttttgatggtttatgccttttcagccatttttgagtttatgcctaacctttttaagcttaaggtttcaacacaactttttgagggtgacaagttcgtaggctcttttgaaaatgctgcagcaggtacatttgtgcttgatatgcatgcctatgagagacttgccaactctatctcaactgaaaaactgcgtcagtatgctagtacttacaataagtacaagtattattcaggcagtgcttcagaggctgattacaggcttgcttgttttgcccatttggccaaggctatgatggattatgcttctaatcacaacgacacgttatacacaccacccactgtgagttacaattcaactctacaggctggcttgcgtaagatggcacaaccatctggtgttgttgagaagtgcatagttcgtgtttgctatggtaatatggctcttaatggcctatggcttggtgatactgttatctgcccacgccatgttatagcgtctagtactactagcactatagattatgactatgccctttctgttttacgcctccacaacttctccatttcatctggtaatgttttcctaggtgttgtgggtgtaaccatgcgaggtgctttgttgcagataaaggttaatcaaaacaatgtccacacgcctaagtacacctatcgcacagttagaccgggtgaatcttttaatatcttggcgtgctatgatggttctgcagctggtgtttacggcgttaacatgcgctctaattacactattagaggctcgttcattaatggcgcttgtggttcacctggttataacattaacaatggtaccgttgagttttgctatttacaccagcttgaacttggttcaggctgtcatgttggtagcgacttagatggtgttatgtatggtggttatgaggaccaacctactttgcaagttgaaggcgctagtagtctgtttacagagaatgtgttggcatttctttatgcagcactcattaatggttctacctggtggcttagttcttctaggattgctgtagacaggtttaatgagtgggctgttcataatggtatgacaacagtagttaatactgattgcttttctattcttgctgctaagactggtgttgatgtacaacgtttgttggcctcaatccagtctctgcataagaattttggtggaaagcaaattcttggctatacctcgttgacagatgagtttactacaggtgaagttatacgtcaaatgtatggcgttaatcttcagagtggttatgtttcacgcgcctgtagaaatgtcttgctggttggttcttttctgactttcttttggtcagaattagtttcctacactaagttcttttgggtaaatcctggttatgtcacacctatgtttgcgtgtttgtcattgctgtcctcacttttgatgttcacactcaagcataagacattgtttttccaggtctttctaatacctgctctgattgttacatcttgcattaatttggcatttgatgttgaagtctacaactatttggcagagcattttgattaccatgtttctctcatgggttttaatgcacaaggtcttgttaacatctttgtctgctttgttgttaccattttacacggcacatacacatggcgcttttttaacacacctgtgagttctgtcacttatgtggtagctttgctgactgcggcatataactatttttacgctagtgacattcttagttgtgctatgacactatttgctagtgtgactggcaactggttcgttggtgctgtttgttataaagctgctgtttatatggccttgagatttcctacttttgtggctatttttggtgatattaagagtgttatgttctgttaccttgtgttgggttattttacctgttgcttctacggtattctctactggttcaacaggttttttaaggttagtgtaggtgtctatgactatactgttagtgctgctgagtttaagtatatggttgctaacggcctacgtgcaccaactggaacacttgattcactacttctgtctgccaaattgattggtattggtggtgagcggaatattaagatttcttccgttcagtctaaactgactgatattaagtgtagtaacgttgtgcttttaggctgtctctctagcatgaatgtctcagcaaattcaacagaatgggcctattgtgttgacttgcataacaagatcaacttgtgtaatgacccagaaaaagcgcaggaaatgctacttgctttgttggcatttttccttagtaagaatagtgcttttggtttagatgacttattggaatcctattttaatgacaatagtatgttgcagagtgttgcatctacttatgtcggtttgccttcttatgtcatttatgaaaatgcacgccaacagtatgaagatgctgttaataatggttctccacctcagttggttaagcaattgcgccatgccatgaatgtagcaaagagcgaatttgaccgtgaggcttctactcagcgtaagcttgatagaatggcggaacaggctgcagcacagatgtacaaagaggcacgagcagttaataggaagtccaaagttgtaagtgctatgcattcactgctttttggtatgttgagacgtttggacatgtcttctgtagacaccattctcaacttggcaaaggatggggttgtacctctgtctgtcataccggcagtcagtgctactaagcttaacattgttacttctgatatcgattcttataatcgtatccagcgtgagggatgtgtccactacgctggtaccatttggaatataattgatatcaaggacaatgatggcaaggtggtacacgttaaggaggtaaccgcacagaatgctgagtccctgtcatggcccctggtccttgggtgtgagcgtattgtcaagctccagaataatgaaattattcccggtaagctgaagcagcgctccattaaggcagaaggagatggcatagttggagaaggtaaggcactttacaataatgagggtggacgtacttttatgtatgctttcatctcggacaaaccggacctgcgtgtagtcaagtgggagttcgatggtggttgtaacactattgagctagaaccaccacgtaagttcttggtggattctcctaatggtgcacagatcaagtatctctactttgttcgtaaccttaacacgttacgtaggggtgctgttctcggctacataggtgccactgtacgcttgcaggctggtaaacaaacagaacaggctattaactcttcattgttgacactttgcgctttcgctgtggatcctgctaagacctacatcgatgctgtcaaaagtggtcacaaaccagtaggtaactgtgttaagatgttggccaatggttctggtaatggacaagctgttactaatggtgtggaggctagtactaaccaggattcatacggtggtgcgtccgtgtgtctatattgtagagcacatgttgagcatccatctatggatggtttttgcagactgaaaggcaagtacgtacaggttccactaggtacagtggatcctatacgttttgtacttgagaatgacgtttgcaaggtttgtggttgttggctggctaatggctgcacttgtgacagatccattatgcaaagcactgatatggcttatttaaacgagtacggggctctagtgcagctcgactagagccctgtaacggtactgatacacaacatgtgtatcgtgcttttgacatctacaacaaggatgttgcttgtctaggtaaattcctcaaggtgaactgtgttcgcctgaagaatttggataagcatgatgcattctatgttgtcaaaagatgtaccaagtctgcgatggaacacgagcaatccatctatagcagacttgaaaagtgtggagccgtagccgaacacgatttcttcacttggaaggatggtcgtgccatctatggtaacgtttgtagaaaggatcttaccgagtatactatgatggatttgtgttacgctttacgtaactttgatgaaaacaattgcgatgttcttaagagcattttaattaaggtaggcgcttgtgaggagtcctacttcaataataaagtctggtttgaccctgttgaaaatgaagacattcatcgtgtctatgcattgttaggtaccattgtttcacgtgctatgcttaaatgcgttaagttctgtgatgcaatggttgaacaaggtatagttggtgttgtcacattagataatcaggatcttaatggtgatttttatgattttggtgattttacttgtagcatcaagggaatgggtatacccatttgcacatcatattactcttatatgatgcctgttatgggtatgactaattgccttgctagtgagtgttttgttaagagtgatatatttggtgaggatttcaagtcatatgacctgctggaatatgatttcacggagcataagacagcactcttcaacaagtatttcaagtattggggactgcaataccaccctaactgtgtggactgcagtgatgagcagtgcatagttcactgtgccaacttcaatacgttgttttccactactatacctattacggcatttggacctttgtgtcgcaagtgttggattgatggtgttccactggtaactacagctggttatcattttaaacagttaggtatagtttggaacaatgacctcaacttacactctagcaggctctctattaacgaattactccagttttgtagtgatcctgcattgcttatagcatcatcaccagcccttgttgatcagcgtactgtttgcttttcagttgcagcgctaggtacaggtatgactaaccagactgttaaacctggccatttcaataaggagttttatgacttcttacttgagcaaggtttcttttctgagggctctgagcttactttaaagcacttcttctttgcacagaagggtgatgcagctgttaaggattttgactactataggtataatagacctactgttctggacatttgccaagctcgcgtcgtgtatcaaatagtgcaacgctattttgatatttacgaaggtggttgtatcactgctaaagaggtggttgttacaaaccttaacaagagcgcaggttatcctttgaacaagtttggtaaagctggtctttactatgagtctttatcctatgaggaacaggatgaactttatgcttatactaagcgtaacatcctgcccactatgacacagctcaaccttaaatatgctataagtggcaaagaacgtgcacgcacagtgggtggtgtttcgcttttgtcaaccatgactactcggcagtatcatcagaaacaccttaagtccatagttaatactaggggcgcttcggttgttattggtactactaagttttatggtggttgggacaatatgcttaagaaccttattgatggtgttgaaaatccgtgtcttatgggttgggactacccaaagtgcgacagagcactgcccaatatgatacgtatgatttcagccatgattttaggctctaagcacaccacatgctgcagttccactgaccgctttttcaggttgtgcaatgaattggctcaagtccttactgaggttgtttattctaatggaggtttttatttgaagccaggtggtactacctctggtgatgcaaccaccgcatatgcaaactcagtttttaatatcttccaagcagtaagtgccaatgttaacaaacttcttagtgttgacagcaatgtctgtcataatttagaagttaagcaattgcagcgtaagctttatgagtgctgttatagatcaactaccgtcgatgaccagttcgtcgttgagtattatggttacttgcgtaaacatttttcaatgatgattctttctgatgatggcgttgtttgttataacaatgactatgcatcacttggttatgtcgctgatcttaacgcattcaaggctgttttgtattaccagaacaatgtcttcatgagcgcctctaaatgttggatcgagcctgacattaataaaggtcctcatgaattttgctcgcagcatactatgcagattgtcgataaagatggtacttattaccttccttaccctgatccttcaagaattctctctgcaggtgtgtttgttgatgacgttgttaaaactgatgcagttgtattgcttgaacgttatgtgtcattggctatagatgcctacccgttatctaagcatgaaaaccctgaatataagaaggtgttttatgtgcttttggattgggttaagcatctgtacaaaactcttaatgctggtgtgttagagtctttttctgtcacacttttggaagattctactgctaaattctgggatgagagcttttatgccaacatgtatgagaaatctgcagttttacaatctgcagggctttgtgttgtttgtggctctcaaactgttttacgttgtggtgattgtctacggcgtcctatgctttgtactaagtgtgcttatgatcatgtcattggaacaactcacaagttcattttggccatcactccatatgtgtgttgtgcttcagattgtggtgtcaatgatgtaactaagctctacttaggtggtcttagttattggtgtcatgaccacaagccacgtcttgcattcccgttgtgctctgctggtaatgtttttggcttgtacaaaaattctgctaccggctcacccgatgttgaagactttaatcgcattgctacatccgattggactgatgtttctgactacaggttggcaaatgatgtcaaggactcattgcgtctgtttgcagcggaaactatcaaggccaaggaggagagcgttaagtcatcctatgcttgtgcaacactacatgaggttgtaggacctaaagagttgttgctcaaatgggaagtcggcagacccaaaccaccccttaatagaaattcggttttcacttgttatcatataacgaagaacaccaaatttcaaatcggtgagtttgtgtttgagaaggcagaatatgataatgatgctgtaacatataaaactaccgccacaacaaaacttgttcctggcatggtttttgtgcttacctcacataatgttcagccattgcgcgcaccgaccattgctaatcaagaacgttattccactatacataagttgcatcctgcttttaacatacctgaagcttattctagcttagtgccctattaccaattgattggtaagcagaagattacaactattcagggacctcccggtagtggtaaatctcactgtgttatagggctaggtttgtactatccaggtgcacgtatagtgtttacagcttgttctcatgcagcggtcgattcactttgtgtgaaagcttccactgcttatagcaatgacaaatgttcacgcatcataccacagcgcgctcgtgttgagtgttatgatggtttcaagtctaataatactagtgctcagtaccttttctctactgtcaatgctttgccagagtgcaatgcggacattgttgtggtggatgaggtctctatgtgcactaattatgacttgtctgtcataaatcagcgcatcagctataggcatgtagtctatgttggtgaccctcaacagctgcctgcaccacgtgttatgatttcacgtggtactttggaaccaaaggactacaacgttgtcactcaacgcatgtgtgcccttaagcctgatgttttcttgcacaagtgttatcgctgtcctgctgagatagtgcgtactgtgtctgagatggtctatgaaaaccaattcattcctgtgcacccagatagcaagcagtgttttaaaatcttttgcaagggtaatgttcaggttgataatggttcaagcattaatcgcaggcaattggatgttgtgcgtatgtttttggctaaaaatcctaggtggtcaaaggctgtttttatttctccttataacagccagaattatgttgccagccgcatgctaggtctacaaattcagacagttgactcatcccagggtagtgagtatgactatgtcatttacacacaaacttcagatactgcccatgcctgtaatgttaacaggtttaatgttgccatcacaagggccaagaaaggcatattatgtataatgtgcgataggtccctttttgatgtgcttaaattctttgagcttaaattgtctgatttgcaggctaatgagggttgtggtctttttaaagactgtagcagaggtgatgatctgttgccaccatctcacgctaacaccttcatgtctttagcggacaattttaagactgatcaagatcttgctgttcaaataggtgttaatggacccattaaatatgagcatgttatctcgtttatgggtttccgttttgatatcaacatacccaaccatcatactctcttttgcacacgcgactttgccatgcgcaatgttagaggttggttaggctttgacgttgaaggagcacatgttgttggctctaacgtcggtacaaatgtcccattgcaattagggttttctaacggtgttgattttgttgtcagacctgaaggttgcgttgtaacagagtctggtgactacattaaacccgtcagagctcgtgctccaccaggggaacaattcgcacaccttttgcctttacttaaacgcggccaaccatgggatgttgtccgcaaacgtatagtgcagatgtgtagtgactacctggccaacctatcagacatactaatttttgtgttgtgggctggtggtttggagttgacaactatgcgttattttgtcaagattggaccaagtaagagttgtgattgtggtaaggttgctacttgttacaatagtgcgctgcatacgtactgttgtttcaaacatgcccttggttgtgattatctgtataacccatactgtattgatatacagcagtggggatacaagggatcacttagccttaaccaccatgagcattgtaatgtacatagaaacgagcatgtggcttctggtgatgccataatgactcgctgtctggccatacatgattgctttgtcaagaacgttgactggtccatcacatacccatttattggtaatgaggctgttattaataagagcggccgaattgtgcaatcacacactatgcggtcagttcttaagttatacaatccgaaagccatatatgatattggcaatcctaagggcattagatgtgccgtaacggatgctaagtggttttgctttgacaagaatcctactaattctaatgtcaagacattggagtatgactatataacacatggccaatttgatgggttgtgcttgttttggaattgcaatgtagacatgtatccagaattttctgtggtctgtcgttttgatactcgctgtaggtcaccactcaacttggagggttgtaatggtggttcactgtatgttaataatcatgcattccatacaccggcttttgacaagcgtgcttttgctaagttgaagccaatgccatttttcttttatgatgatactgagtgtgacaagttacaggactccataaactatgttcctcttagggctagtaactgcattactaaatgtaatgttggtggtgctgtctgtagtaagcattgtgctatgtatcatagctatgttaatgcttacaacacttttacgtcggcgggctttactatttgggtgcctacttcgtttgacacctataatctgtggcagacatttagtaacaatttgcaaggtcttgagaacattgctttcaatgtcgtaaagaaaggatcttttgttggtgccgaaggtgaacttcctgtagctgtggttaatgacaaagtgctcgttagagatggtactgttgatactcttgtttttacaaacaagacatcactacccactaacgtagcttttgagttgtatgccaagcgtaaggtaggactcaccccacccattacgatcctacgtaacttgggtgtagtttgtacatctaagtgtgtcatttgggactatgaagccgaacgtccacttactacttttacaaaggatgtttgtaaatataccgactttgagggtgacgtctgtacactctttgataacagcattgttggttcattagagcgattctccatgacccaaaatgctgtgcttatgtcacttacagctgttaaaaagcttactggcataaagttaacttatggttatcttaatggtgtcccagttaacacacatgaagataaaccttttacttggtatatttacactaggaagaacggcaagttcgaggaccatcctgatggctattttacccaaggtagaacaaccgctgattttagccctcgtagcgacatggaaaaggacttcctaagtatggatatgggtctgtttattaacaagtacggacttgaagattacggctttgagcacgttgtgtatggtgatgtttcaaaaaccacccttggtggtttgcatctactaatttcgcaggtgcgtctggcctgtatgggtgtgctcaaaatagacgagtttgtgtctagtaatgatagcacgttaaagtcttgtactgttacatatgctgataaccctagtagtaagatggtttgtacgtatatggatctcctgcttgacgattttgtcagcattcttaaatctttggatttgggcgttgtatctaaagttcatgaagttatggtcgattgtaaaatgtggaggtggatgttgtggtgtaaggatcataaactccagacattttatccgcaacttcaggccagtgaatggaagtgtggttattccatgccttctatttacaagatacaacgtatgtgtttagaaccttgcaatctctacaactatggtgctggtattaagttacctgatggcattatgtttaacgtagttaaatacacacagctttgtcaatatctcaatagcaccacaatgtgtgtaccccatcacatgcgtgtgctacatcttggtgctggctccgacaagggtgttgcacctggcacggctgtcttacgacgttggttgccactggatgccattatagttgacaatgatagtgtggattacgttagcgatgctgattatagtgttacaggagattgctctaccttatacctgtcagataagtttgatttagttatatctgatatgtatgatggtaagattaaaagttgtgatggggagaacgtgtctaaagaaggcttctttccctatattaatggtgtcatcaccgaaaagttggcacttggtggtactgtagctattaaggtgacggagtttagttggaataagaagttgtatgaactcattcagaggtttgagtattggacaatgttctgtaccagtgttaacacgtcatcgtcagaggcattcttaattggtgttcactatttaggtgattttgcaagtggcgctgtgattgacggcaacactatgcatgccaattatatcttctggcgtaattccacaattatgactatgtcttacaatagtgtacttgatttaagcaagttcaattgtaagcataaggctacagttgtcattaatttaaaagattcatccattagtgatgttgtgttaggtttgttgaagaatggtaagttgctagtgcgtaataatgacgccatttgtggtttttctaatcatttggtcaacgtaaacaaatgaagtctttaacctacttctggttgttcttaccagtactttcaacacttagcctaccacaagatgtcaccaggtgctcagctaacactaattttaggcggttcttttcaaaatttaatgttcaggcgcctgcagttgttgtactgggcggttatctacctattggtgaaaaccagggtgtcaattcaacttggtactgtgctggccaacatccaactgctagtggcgttcatggtatctttgttagccatattagaggtggtcatggctttgagattggcatttcgcaagagccttttgaccctagtggttaccagctttatttacataaggctactaacggtaacactaatgctactgcgcgactgcgcatttgccagtttcctagcattaaaacattgggccccactgctaataatgatgttacaacaggtcgtaattgcctatttaacaaagccatcccagctcatatgagtgaacatagtgttgtcggcataacatgggataatgatcgtgtcactgtcttttctgacaagatctattatttttattttaaaaatgattggtcccgtgttgcgacaaagtgttacaacagtggaggttgtgctatgcaatatgtttacgaacccacctattacatgcttaatgttactagtgctggtgaggatggtatttcttatcaaccctgtacagctaattgcattggttatgctgccaatgtatttgctactgagcccaatggccacataccagaaggttttagttttaataattggtttcttttgtccaatgattccactttggtgcatggtaaggtggtttccaaccaaccattgttggtcaattgtcttttggccattcctaagatttatggactaggccaatttttctcctttaatcaaacgatcgatggtgtttgtaatggagctgctgtgcagcgtgcaccagaggctctgaggtttaatattaatgacatctctgtcattcttgctgaaggctcaattgtacttcatactgctttaggaacaaatttttcttttgtttgcagtaattcctcaaatcctcacttagccaccttcgccatacctctgggtgctacccaagtaccttattattgtttttttaaagtggatacttacaactccactgtttataaatttttggctgttttacctcctaccgtcagggaaattgtcatcaccaagtatggtgatgtttatgtcaatgggtttggatacttgcatctcggtttgttggatgctgtcacaattaatttcactggtcatggcactgacgatgatgtttctggtttttggaccatagcatcgactaattttgttgatgcactcatcgaagttcaaggaaccgccattcagcgtattctttattgtgatgatcctgttagccaactcaagtgttctcaggttgcttttgaccttgacgatggtttttacactatttcttctagaaaccttctgagtcatgaacagccaatttcttttgttactctgccatcatttaatgatcattcttttgttaacattactgtatctgcttcctttggtggtcatagtggtgccaaccttattgcatctgacactactatcaatgggtttagttctttctgtgttgacactagacaatttaccatttcactgttttataacgttacaaacagttatggttatgtgtctaaatcacaggacagtaattgccctttcaccttgcaatctgttaatgattacctgtcttttagcaaattttgtgtttccaccagccttttggctagtgcctgtaccatagatctttttggttaccctgagtttggtagtggtgttaagtttacgtccctttactttcaattcacaaagggtgagttgattactggcacgcctaaaccacttgaaggtgtcacggacgtttcttttatgactctggatgtgtgtaccaagtatactatctatggctttaaaggtgagggtatcattacccttacaaattctagctttttggcaggtgtttattacacatctgattctggacagttgttagcctttaagaatgtcactagtggtgctgtttattctgttacgccatgttctttttcagagcaggctgcatatgttgatgatgatatagtgggtgttatttctagtttgtctagctccacttttaacagtactagggagttgcctggtttcttctaccattctaatgatggctctaattgtacagagcctgtgttggtgtatagtaacataggtgtttgtaaatctggcagtattggctacgtcccatctcagtctggccaagtcaagattgcacccacggttactgggaatattagtattcccaccaactttagtatgagtattaggacagaatatttacagctttacaacacgcctgttagtgttgattgtgccacatatgtttgtaatggtaactctcgttgtaaacaattactcacccagtacactgcagcatgtaagaccatagagtcagcattacaactcagcgctaggcttgagtctgttgaagttaactctatgcttactatttctgatgaggctctacagttagctaccattagttcgtttaatggtgatggatataattttactaatgtgctgggtgtttctgtgtatgatcctgcaagtggcagggtggtacaaaaaaggtcttttattgaagacctgctttttaataaagtggttactaatggccttggtactgttgatgaagactataagcgctgttctaatggtcgctctgtggcagatctagtctgtgcacagtattactctggtgtcatggtactacctggtgttgttgacgctgagaagcttcacatgtatagtgcgtctctcatcggtggtatggtgctaggaggttttacttctgcagcggcattgccttttagctatgctgttcaagctagactcaattatcttgctctacagacggatgttctacagcggaaccagcaattgcttgctgagtcttttaactctgctattggtaatataacttcagcctttgagagtgttaaagaggctattagtcaaacttccaagggtttgaacactgtggctcatgcgcttactaaggttcaagaggttgttaactcgcagggtgcagctttgactcaacttaccgtacagctgcaacacaacttccaagccatttctagttctattgatgacatttactctcgactggacattctttcagccgatgctcaggttgaccgtctcatcaccggcagattatcagcacttaatgcttttgttgctcaaaccctcactaagtatactgaggttcaggctagcaggaagttagcacagcaaaaggttaatgagtgcgttaaatcgcaatctcagcgttatggtttttgtggtggtgatggcgagcacattttctctctggtacaggcagcacctcagggcctgctgtttttacatacagtacttgtaccgagtgattttgtagatgttattgccatcgctggcttatgcgttaacgatgaaattgccttgactctacgtgagcctggcttagtcttgtttacgcatgaacttcaaaatcatactgcgacggaatattttgtttcatcgcgacgtatgtttgaacctagaaaacctaccgttagtgattttgttcaaattgagagttgtgtggtcacctatgtcaatttgactagagaccaactaccagatgtaatcccagattacatcgatgttaacaaaacactttatgagattttagcttctctgcccaatagaactggtccaagtcttcctttagatgtttttaatgccacttatcttaatctcactggtgaaattgcagatttagagcagcgttcagagtctctccgtaatactacagaggagctccaaagtcttatatataatatcaacaacacactagttgaccttgagtggctcaaccgagttgagacatatatcaagtggccgtggtgggtttggttgattattttcattgttctcatctttgttgtgtcattactagtgttctgctgcatttccacgggttgttgtggatgctgcggctgctgctgtgcttgtttctcaggttgttgtaggggtcctagacttcaaccttacgaagtttttgaaaaggtccacgtgcagtgatgtttcttggactttttcaatacacgattgacacagttgtcaaagatgtctcaaagtctgctaacttgtctttggatgctgtccaagagttggagctcaatgtagttccaattagacaagcttcaaatgtgacgggttttcttttcaccagtgtttttatctacttctttgcactgtttaaagcgtcttctttgaggcgcaattatattatgttggcagcgcgttttgctgtcattgttctttattgcccacttttatattattgtggtgcatttttagatgcaactattatttgttgcacacttattggcaggctttgtttagtctgcttttactcctggcgctataaaaatgcgctctttattatttttaatactacgacactttctttcctcaatggtaaagcagcttattatgacggcaaatccattgtgattttagaaggtggtgaccattacatcacttttggcaactcttttgttgcttttgttagtagcatcgacttgtatctagctatacgtgggcggcaagaagctgacctacagctgttgcgaactgttgagcttcttgatggcaagaagctttatgtcttttcgcaacatcaaattgttggcattactaatgctgcatttgactcaattcaactagacgagtatgctacaattagtgaatgataatggtctagtagttaatgttatactttggcttttcgtactctttttcctgcttattataagcattactttcgtccaattggttaatctgtgcttcacttgtcaccggttgtgtaatagcgcagtttacacacctatagggcgtttgtatagagtttataagtcttacatgcaaatagaccccctccctagtactgttattgacgtataaacgaaatatgtctaacggttctattcccgttgatgaggtgattcaacaccttagaaactggaatttcacatggaatatcatactgacgatactacttgtagtgcttcagtatggccattacaagtactctgcgttcttgtatggtgtcaagatggctattctatggatactttggcctcttgtgttagcactgtcactttttgatgcatgggctagctttcaggtcaattgggtcttttttgctttcagcatccttatggcttgcatcactcttatgctgtggataatgtactttgtcaatagcattcggttgtggcgcaggacacattcttggtggtctttcaatcctgaaacagacgcgcttctcactacttctgtgatgggccgacaggtctgcattccagtgcttggagcaccaactggtgtaacgctaacactccttagtggtacattgcttgtagagggctataaggttgctactggcgtacaggtaagtcaattacctaatttcgtcacagtcgccaaggccactacaacaattgtctacggacgtgttggtcgttcagtcaatgcttcatctggcactggttgggctttctatgtccggtccaaacacggcgactactcagctgtgagtaatccgagttcggttctcacagatagtgagaaagtgcttcatttagtctaaacagaaactttatggcttctgtcagttttcaggatcgtggccgcaaacgggtgccattatccctctatgcccctcttagggttactaatgacaaacccctttctaaggtacttgcaaataatgctgtacccactaataaaggaaataaggaccagcaaattggatactggaatgagcaaattcgctggcgcatgcgccgtggtgagcgaattgaacaaccttccaattggcatttctactacctcggaacaggacctcacgccgacctccgctataggactcgtactgagggtgttttctgggttgctaaagaaggcgcaaagactgaacccactaacctgggtgtcagaaaggcgtctgaaaagccaattattccaaatttctctcaacagcttcccagcgtagttgagattgttgaacctaacacacctcctacttcacgtgcaaattcacgtagcaggagtcgtggtaatggcaacaacaggtccagatctccaagtaacaacagaggcaataaccagtcccgcggtaattcacagaatcgtggaaataaccagggtcgtggagcttctcagaacagaggaggcaataataataacaataacaagtctcgtaaccagtccaagaacagaaaccagtcaaatgaccgtggtggtgtaacatcacgcgatgatctggtggctgctgtcaaggatgcccttaaatctttgggtattggcgaaaaccctgacaagcttaagcaacagcagaagcccaaacaggaaaggtctgacagcagcggcaaaaatacacctaagaagaacaaatccagagccacttcgaaagaacgtgacctcaaagacatcccagagtggaggagaattcccaagggcgaaaatagcgtagcagcttgcttcggacccaggggaggcttcaaaaattttggagatgcggaatttgtcgaaaaaggtgttgatgcctcaggctatgctcagatcgccagtttagcaccaaatgttgcagcattgctctttggtggtaatgtggctgttcgtgagctagcggactcttacgagattacatataattataaaatgactgtgccaaagtctgatccaaatgtagagcttcttgtttcacaggtggatgcatttaaaactgggaatgcaaaaccccagagaaagaaggaaaagaagaacaagcgtgaaaccacgcagcagctgaatgaagaggccatctacgatgatgtgggtgtgccatctgatgtgactcatgccaatttggaatgggacacagctgttgatggtggtgacacggccgttgaaattatcaacgagatcttcgacacaggaaattaaacaatgtttgactggcttatcctggctatgtcccagggtagtgccattacactgttattactgagtgtttttctagcgacttggctgctgggctatggctttgccctctaactagcggtcttggtcttgcacacaacggtaagccagtggtaatgtcagtgcaagaaggatattaccatagcactgtcatgaggggaacgcagtaccttttcatctaaacctttgcacgagtaatcaaagatccgcttgacgagcctatatggaagagcgtgccaggtatttgactcaaggactgttagtaactgaagacctgacggtgttgatatggatacac

SEQ ID NO: 2 - DNA, 20 bp, PEDV
acgtccgtaacaccttcaag

SEQ ID NO: 3 - DNA, 22 bp, PEDV
gctagtgcctgtaccatagatc

SEQ ID NO: 4 - DNA, 27 bp, PEDV
cgtgccagtaatcaactcaccctttgt

SEQ ID NO: 5 - protein, 76 aa, PEDV
MLQLVNDNGLVVNVILWLFVLFFLLIISITFVQLVNLCFTCHRLCNSAVYTPIGRLYRVYKSYMQIDPLPSTVIDV

SEQ ID NO: 6 - protein, 226 aa, PEDV
MSNGSIPVDEVIQHLRNWNFTWNIILTILLVVLQYGHYKYSAFLYGVKMAILWILWPLVLALSLFDAWASFQVNWVFFAFSILMACITLMLWIMYFVNSIRLWRRTHSWWSFNPETDALLTTSVMGRQVCIPVLGAPTGVTLTLLSGTLLVEGYKVATGVQVSQLPNFVTVAKATTTIVYGRVGRSVNASSGTGWAFYVRSKHGDYSAVSNPSSVLTDSEKVLHLV

SEQ ID NO: 7 - protein, 441 aa, PEDV
MASVSFQDRGRKRVPLSLYAPLRVTNDKPLSKVLANNAVPTNKGNKDQQIGYWNEQIRWRMRRGERIEQPSNWHFYYLGTGPHADLRYRTRTEGVFWVAKEGAKTEPTNLGVRKASEKPIIPNFSQQLPSVVEIVEPNTPPTSRANSRSRSRGNGNNRSRSPSNNRGNNQSRGNSQNRGNNQGRGASQNRGGNNNNNNKSRNQSKNRNQSNDRGGVTSRDDLVAAVKDALKSLGIGENPDKLKQQQKPKQERSDSSGKNTPKKNKSRATSKERDLKDIPEWRRIPKGENSVAACFGPRGGFKNFGDAEFVEKGVDASGYAQIASLAPNVAALLFGGNVAVRELADSYEITYNYKMTVPKSDPNVELLVSQVDAFKTGNAKPQRKKEKKNKRETTQQLNEEAIYDDVGVPSDVTHANLEWDTAVDGGDTAVEIINEIFDTGN

SEQ ID NO: 8 - protein, 6725 aa, PEDV
MASNHVTLAFANDAEISAFGFCTASEAVSYYSEAAASGFMQCRFVSFDLADTVEGLLPEDYVMVVVGTTKLSAYVDTFGSRPKNICGWLLFSNCNYFLEELELTFGRRGGNIVPVDQYMCGADGKPVLQESEWEYTDFFADSEDGQLNIAGITYVKAWIVERSDVSYASQNLTSIKSITYCSTYEHTFPDGTAMKVARTPKIKKTVVLSEPLATIYREIGSPFVDNGSDARSIIKRPVFLHAFVKCKCGSYHWTVGDWTSYVSTCCGFKCKPVLVASCSATPGSVVVTRAGAGTGVKYYNNMFLRHVADIDGLAFWRILKVQSKDDLACSGKFLEHHEEGFTDPCYFLNDSSIATKLKFDILSGKFSDEVKQAIFAGHVVVGSALVDIVDDALGQPWFIRKLGDLASAAWEQLKAVVRGLNLLSDEVVLFGKRLSCATLSIVNGVFEFIAEVPEKLAAAVTVFVNFLNELFESACDCLKVGGKTFNKVGSYVLFDNALVKLVKAKVRGPRQAGVCEVRYTSLVIGSTTKVVSKRVENANVNLVVVDEDVTLNTTGRTVVVDGLAFFESDGFYRHLADADVVIEHPVYKSACELKPVFECDPIPDFPMPVAASVAELCVQTDLLLKNYNTPYKTYSCVVRGDKCCITCTLHFTAPSYMEAAANFVDLCTKNIGTAGFHEFYITAHEQQDLQGFVTTCCTMSGFECFMPIIPQCPAVLEEIDGGSIWRSFITGLNTMWDFCKHLKVSFGLDGIVVTVARKFKRLGALLAEMYKTYLSTVVENLVLAGVSFKYYATSVPKIVLGCCFHSVKSVLASAFQIPVQAGVEKFKVFLNCVHPVVPRVIETSFVELEETTFKPPALNGSIAIVDGFAFYYDGTLYYPTDGNSVVPICFKKKGGGDVKFSDEVSVKTIDPVYKVSLEFEFESETIMAVLNKAVGNCIKVTGGWDDVVEYINVAIEVLKDHIDVPKYYIYDEEGGTDPNLPVMVSQWPLNDDTISQDLLDVEVVTDAPVDFEGDEVDSSDPDKVADVANSEPEDDGLNVAPETNVESEVEEVAATLSFIKDTPSTVTKDPFAFDFASYGGLKVLRQSHNNCWVTSTLVQLQLLGIVDDPAMELFSAGRVGPMVRKCYESQKAILGSLGDVSACLESLTKDLHTLKITCSVVCGCGTGERIYDGCAFRMTPTLEPFPYGACAQCAQVLMHTFKSIVGTGIFCRDTTALSLDSLVVKPLCAAAFIGKDSGHYVTNFYDAAMAIDGYGRHQIKYDTLNTICVKDVNWTAPFVPDVEPVLELVVKPFYSYKNVDFYQGDFSDLVKLPCDFVVNAANENLSHGGGIAKAIDVYTKGMLQKCSNDYIKAHGPIKVGRGVMLEALGLKVFNVVGPRKGKHAPELLVKAYKSVFANSGVALTPLISVGIFSVPLEESLSAFLACVGDRHCKCFCYSDKEREAIINYMDGLVDAIFKDALVDTTPVQEDVQQVSQKPVLPNFEPFRIEGAHAFYECNPEGLMSLGADKLVLFTNSNLDFCSVGKCLNNVTGGALLEAINVFKKSNKTVPAGNCVTFECADMISITMVVLPSDGDANYDKNYARAVVKVSKLKGKLLLAVGDAMLYSKLSHLSVLGFVSTPDDVERFYANKSVVIKVTEDTRSVKTVKVESTVTYGQQIGPCLVNDTVVTDNKPVVADVVAKVVPSANWDSHYGFDKAGEFHMLDHTGFAFPSEVVNGRRVLKTTDNNCWVNVTCLQLQFARFRFKSAGLQAMWESYCTGDVAMFVHWLYWLTGVDKGQPSDSENALNMLSKYIVPAGSVTIERVTHDGCCCSKRVVTAPVVNASVLKLGVEDGLCPHGLNYIDKVVVVKGTTIVVNVGKPVVAPSHLFLKGVSYTTFLDNGNGVAGHYTVFDHDTGMVHDGDVFVPGDLNVSPVTNVVVSEQTAVVIKDPVKKVELDATKLLDTMNYASERFFSFGDFMSRNLITVFLYILSILGLCFRAFRKRDVKVLAGVPQRTGIILRKSVRYNAKALGVFFKLKLYWFKVLGKFSLGIYALYALLFMTIRFTPIGGPVCDDVVAGYANSSFDKNEYCNSVICKVCLYGYQELSDFSHTQVVWQHLRDPLIGNVMPFFYLAFLAIFGGVYVKAITLYFIFQYLNILGVFLGLQQSIWFLQLVPFDVFGDEIVVFFIVTRVLMFLKHVFLGCDKASCVACSKSARLKRVPVQTIFQGTSKSFYVHANGGSKFCKKHNFFCLNCDSYGPGCTFINDVIATEVGNVVKLNVQPTGPATILIDKVEFSNGFYYLYSGDTFWKYNFDITDNKYTCKESLKNCSIITDFIVFNNNGSNVNQVKNACVYFSQMLCKPVKLVDSALLASLSVDFGASLHSAFVSVLSNSFGKDLSSCNDMQDCKSTLGFDDVPLDTFNAAVAEAHRYDVLLTDMSFNNFTTSYAKPEEKLPVHDIATCMRVGAKIVNHNVLVKDSIPVVWLVRDFIALSEETRKYIIRTTKVKGITFMLTFNDCRMHTTIPTVCIANKKGAGLPSFSKVKKFFWFLCLFIVAVFFALSFFDFSTQVSSDSDYDFKYIESGQLKTFDNPLSCVHNVFSNFDQWHDAKFGFTPVNNPSCPIVVGVSDEARTVPGIPAGVYLAGKTLVFAINTIFGTSGLCFDASGVADKGACIFNSACTTLSGLGGTAVYCYKNGLVEGAKLYSELAPHSYYKMVDGNAVSLPEIISRGFGIRTIRTKAMTYCRVGQCVQSAEGVCFGADRFFVYNAESGSDFVCGTGLFTLLMNVISVFSKTVPVTVLSGQILFNCIIAFAAVAVCFLFTKFKRMFGDMSVGVFTVGACTLLNNVSYIVTQNTLGMLGYATLYFLCTKGVRYMWIWHLGFLISYILIAPWWVLMVYAFSAIFEFMPNLFKLKVSTQLFEGDKFVGSFENAAAGTFVLDMHAYERLANSISTEKLRQYASTYNKYKYYSGSASEADYRLACFAHLAKAMMDYASNHNDTLYTPPTVSYNSTLQAGLRKMAQPSGVVEKCIVRVCYGNMALNGLWLGDTVICPRHVIASSTTSTIDYDYALSVLRLHNFSISSGNVFLGVVGVTMRGALLQIKVNQNNVHTPKYTYRTVRPGESFNILACYDGSAAGVYGVNMRSNYTIRGSFINGACGSPGYNINNGTVEFCYLHQLELGSGCHVGSDLDGVMYGGYEDQPTLQVEGASSLFTENVLAFLYAALINGSTWWLSSSRIAVDRFNEWAVHNGMTTVVNTDCFSILAAKTGVDVQRLLASIQSLHKNFGGKQILGYTSLTDEFTTGEVIRQMYGVNLQSGYVSRACRNVLLVGSFLTFFWSELVSYTKFFWVNPGYVTPMFACLSLLSSLLMFTLKHKTLFFQVFLIPALIVTSCINLAFDVEVYNYLAEHFDYHVSLMGFNAQGLVNIFVCFVVTILHGTYTWRFFNTPVSSVTYVVALLTAAYNYFYASDILSCAMTLFASVTGNWFVGAVCYKAAVYMALRFPTFVAIFGDIKSVMFCYLVLGYFTCCFYGILYWFNRFFKVSVGVYDYTVSAAEFKYMVANGLRAPTGTLDSLLLSAKLIGIGGERNIKISSVQSKLTDIKCSNVVLLGCLSSMNVSANSTEWAYCVDLHNKINLCNDPEKAQEMLLALLAFFLSKNSAFGLDDLLESYFNDNSMLQSVASTYVGLPSYVIYENARQQYEDAVNNGSPPQLVKQLRHAMNVAKSEFDREASTQRKLDRMAEQAAAQMYKEARAVNRKSKVVSAMHSLLFGMLRRLDMSSVDTILNLAKDGVVPLSVIPAVSATKLNIVTSDIDSYNRIQREGCVHYAGTIWNIIDIKDNDGKVVHVKEVTAQNAESLSWPLVLGCERIVKLQNNEIIPGKLKQRSIKAEGDGIVGEGKALYNNEGGRTFMYAFISDKPDLRVVKWEFDGGCNTIELEPPRKFLVDSPNGAQIKYLYFVRNLNTLRRGAVLGYIGATVRLQAGKQTEQAINSSLLTLCAFAVDPAKTYIDAVKSGHKPVGNCVKMLANGSGNGQAVTNGVEASTNQDSYGGASVCLYCRAHVEHPSMDGFCRLKGKYVQVPLGTVDPIRFVLENDVCKVCGCWLANGCTCDRSIMQSTDMAYLNEYGALVQLDMEHEQSIYSRLEKCGAVAEHDFFTWKDGRAIYGNVCRKDLTEYTMMDLCYALRNFDENNCDVLKSILIKVGACEESYFNNKVWFDPVENEDIHRVYALLGTIVSRAMLKCVKFCDAMVEQGIVGVVTLDNQDLNGDFYDFGDFTCSIKGMGIPICTSYYSYMMPVMGMTNCLASECFVKSDIFGEDFKSYDLLEYDFTEHKTALFNKYFKYWGLQYHPNCVDCSDEQCIVHCANFNTLFSTTIPITAFGPLCRKCWIDGVPLVTTAGYHFKQLGIVWNNDLNLHSSRLSINELLQFCSDPALLIASSPALVDQRTVCFSVAALGTGMTNQTVKPGHFNKEFYDFLLEQGFFSEGSELTLKHFFFAQKGDAAVKDFDYYRYNRPTVLDICQARVVYQIVQRYFDIYEGGCITAKEVVVTNLNKSAGYPLNKFGKAGLYYESLSYEEQDELYAYTKRNILPTMTQLNLKYAISGKERARTVGGVSLLSTMTTRQYHQKHLKSIVNTRGASVVIGTTKFYGGWDNMLKNLIDGVENPCLMGWDYPKCDRALPNMIRMISAMILGSKHTTCCSSTDRFFRLCNELAQVLTEVVYSNGGFYLKPGGTTSGDATTAYANSVFNIFQAVSANVNKLLSVDSNVCHNLEVKQLQRKLYECCYRSTTVDDQFVVEYYGYLRKHFSMMILSDDGVVCYNNDYASLGYVADLNAFKAVLYYQNNVFMSASKCWIEPDINKGPHEFCSQHTMQIVDKDGTYYLPYPDPSRILSAGVFVDDVVKTDAVVLLERYVSLAIDAYPLSKHENPEYKKVFYVLLDWVKHLYKTLNAGVLESFSVTLLEDSTAKFWDESFYANMYEKSAVLQSAGLCVVCGSQTVLRCGDCLRRPMLCTKCAYDHVIGTTHKFILAITPYVCCASDCGVNDVTKLYLGGLSYWCHDHKPRLAFPLCSAGNVFGLYKNSATGSPDVEDFNRIATSDWTDVSDYRLANDVKDSLRLFAAETIKAKEESVKSSYACATLHEVVGPKELLLKWEVGRPKPPLNRNSVFTCYHITKNTKFQIGEFVFEKAEYDNDAVTYKTTATTKLVPGMVFVLTSHNVQPLRAPTIANQERYSTIHKLHPAFNIPEAYSSLVPYYQLIGKQKITTIQGPPGSGKSHCVIGLGLYYPGARIVFTACSHAAVDSLCVKASTAYSNDKCSRIIPQRARVECYDGFKSNNTSAQYLFSTVNALPECNADIVVVDEVSMCTNYDLSVINQRISYRHVVYVGDPQQLPAPRVMISRGTLEPKDYNVVTQRMCALKPDVFLHKCYRCPAEIVRTVSEMVYENQFIPVHPDSKQCFKIFCKGNVQVDNGSSINRRQLDVVRMFLAKNPRWSKAVFISPYNSQNYVASRMLGLQIQTVDSSQGSEYDYVIYTQTSDTAHACNVNRFNVAITRAKKGILCIMCDRSLFDVLKFFELKLSDLQANEGCGLFKDCSRGDDLLPPSHANTFMSLADNFKTDQDLAVQIGVNGPIKYEHVISFMGFRFDINIPNHHTLFCTRDFAMRNVRGWLGFDVEGAHVVGSNVGTNVPLQLGFSNGVDFVVRPEGCVVTESGDYIKPVRARAPPGEQFAHLLPLLKRGQPWDVVRKRIVQMCSDYLANLSDILIFVLWAGGLELTTMRYFVKIGPSKSCDCGKVATCYNSALHTYCCFKHALGCDYLYNPYCIDIQQWGYKGSLSLNHHEHCNVHRNEHVASGDAIMTRCLAIHDCFVKNVDWSITYPFIGNEAVINKSGRIVQSHTMRSVLKLYNPKAIYDIGNPKGIRCAVTDAKWFCFDKNPTNSNVKTLEYDYITHGQFDGLCLFWNCNVDMYPEFSVVCRFDTRCRSPLNLEGCNGGSLYVNNHAFHTPAFDKRAFAKLKPMPFFFYDDTECDKLQDSINYVPLRASNCITKCNVGGAVCSKHCAMYHSYVNAYNTFTSAGFTIWVPTSFDTYNLWQTFSNNLQGLENIAFNVVKKGSFVGAEGELPVAVVNDKVLVRDGTVDTLVFTNKTSLPTNVAFELYAKRKVGLTPPITILRNLGVVCTSKCVIWDYEAERPLTTFTKDVCKYTDFEGDVCTLFDNSIVGSLERFSMTQNAVLMSLTAVKKLTGIKLTYGYLNGVPVNTHEDKPFTWYIYTRKNGKFEDHPDGYFTQGRTTADFSPRSDMEKDFLSMDMGLFINKYGLEDYGFEHVVYGDVSKTTLGGLHLLISQVRLACMGVLKIDEFVSSNDSTLKSCTVTYADNPSSKMVCTYMDLLLDDFVSILKSLDLGVVSKVHEVMVDCKMWRWMLWCKDHKLQTFYPQLQASEWKCGYSMPSIYKIQRMCLEPCNLYNYGAGIKLPDGIMFNVVKYTQLCQYLNSTTMCVPHHMRVLHLGAGSDKGVAPGTAVLRRWLPLDAIIVDNDSVDYVSDADYSVTGDCSTLYLSDKFDLVISDMYDGKIKSCDGENVSKEGFFPYINGVITEKLALGGTVAIKVTEFSWNKKLYELIQRFEYWTMFCTSVNTSSSEAFLIGVHYLGDFASGAVIDGNTMHANYIFWRNSTIMTMSYNSVLDLSKFNCKHKATVVINLKDSSISDVVLGLLKNGKLLVRNNDAICGFSNHLVNVNK

SEQ ID NO: 9 - protein, 222 aa, PEDV
MFLGLFQYTIDTVVKDVSKSANLSLDAVQELELNVVPIRQASNVTGFLFTSVFIYFFALFKASSLRRNYIMLAARFAVIVLYCPLLYYCGAFLDATIICCTLIGRLCLVCFYSWRYKNALFIIFNTTTLSFLNGKAAYYDGKSIVILEGGDHYITFGNSFVAFVSSIDLYLAIRGRQEADLQLLRTVELLDGKKLYVFSQHQIVGITNAAFDSIQLDEYATI

SEQ ID NO: 10 - protein, 1386 aa, PEDV
MKSLTYFWLFLPVLSTLSLPQDVTRCSANTNFRRFFSKFNVQAPAVVVLGGYLPIGENQGVNSTWYCAGQHPTASGVHGIFVSHIRGGHGFEIGISQEPFDPSGYQLYLHKATNGNTNATARLRICQFPSIKTLGPTANNDVTTGRNCLFNKAIPAHMSEHSVVGITWDNDRVTVFSDKIYYFYFKNDWSRVATKCYNSGGCAMQYVYEPTYYMLNVTSAGEDGISYQPCTANCIGYAANVFATEPNGHIPEGFSFNNWFLLSNDSTLVHGKVVSNQPLLVNCLLAIPKIYGLGQFFSFNQTIDGVCNGAAVQRAPEALRFNINDISVILAEGSIVLHTALGTNFSFVCSNSSNPHLATFAIPLGATQVPYYCFFKVDTYNSTVYKFLAVLPPTVREIVITKYGDVYVNGFGYLHLGLLDAVTINFTGHGTDDDVSGFWTIASTNFVDALIEVQGTAIQRILYCDDPVSQLKCSQVAFDLDDGFYTISSRNLLSHEQPISFVTLPSFNDHSFVNITVSASFGGHSGANLIASDTTINGFSSFCVDTRQFTISLFYNVTNSYGYVSKSQDSNCPFTLQSVNDYLSFSKFCVSTSLLASACTIDLFGYPEFGSGVKFTSLYFQFTKGELITGTPKPLEGVTDVSFMTLDVCTKYTIYGFKGEGIITLTNSSFLAGVYYTSDSGQLLAFKNVTSGAVYSVTPCSFSEQAAYVDDDIVGVISSLSSSTFNSTRELPGFFYHSNDGSNCTEPVLVYSNIGVCKSGSIGYVPSQSGQVKIAPTVTGNISIPTNFSMSIRTEYLQLYNTPVSVDCATYVCNGNSRCKQLLTQYTAACKTIESALQLSARLESVEVNSMLTISDEALQLATISSFNGDGYNFTNVLGVSVYDPASGRVVQKRSFIEDLLFNKVVTNGLGTVDEDYKRCSNGRSVADLVCAQYYSGVMVLPGVVDAEKLHMYSASLIGGMVLGGFTSAAALPFSYAVQARLNYLALQTDVLQRNQQLLAESFNSAIGNITSAFESVKEAISQTSKGLNTVAHALTKVQEVVNSQGAALTQLTVQLQHNFQAISSSIDDIYSRLDILSADAQVDRLITGRLSALNAFVAQTLTKYTEVQASRKLAQQKVNECVKSQSQRYGFCGGDGEHIFSLVQAAPQGLLFLHTVLVPSDFVDVIAIAGLCVNDEIALTLREPGLVLFTHELQNHTATEYFVSSRRMFEPRKPTVSDFVQIESCVVTYVNLTRDQLPDVIPDYIDVNKTLYEILASLPNRTGPSLPLDVFNATYLNLTGEIADLEQRSESLRNTTEELQSLIYNINNTLVDLEWLNRVETYIKWPWWVWLIIFIVLIFVVSLLVFCCISTGCCGCCGCCCACFSGCCRGPRLQPYEVFEKVHVQ

(19) United States
(12) Patent Application Publication
(10) Pub. No.: US 2016/0008457 A1
(43) Pub. Date: Jan. 14, 2016

(54) Inactivated Vaccine for Porcine Epidemic Diarrhea Virus (PEDV)

(71) Applicant: MERIAL, INC., DULUTH, GA (US)

(72) Inventors: Srivishnupriya Anbalagan, Vermillion, SD (US); Emily Collin, Windom, MN (US); Benjamin Matthew Hause, Currie, MN (US)

(73) Assignee: MERIAL, INC., DULUTH, GA (US)

(21) Appl. No.: 14/798,132

(22) Filed: Jul. 13, 2015

Related U.S. Application Data
(60) Provisional application No. 62/023,434, filed on Jul. 11, 2014.

(51) Int. Cl. A61K 39/215; C12N 7/00

(52) U.S. Cl. CPC A61K 39/215; C12N 7/00; C12N 2770/18034; C12N 2770/18071; A61K 2039/5252

(57) ABSTRACT

The present invention encompasses porcine epidemic diarrhea virus (PEDV) vaccines or compositions. The vaccine or composition may be a vaccine or composition containing inactivated PEDV. The invention also encompasses epitopes or immunogens which can be used to protect porcine animals against PEDV.

### INCORPORATION BY REFERENCE

This application claims priority to provisional application U.S. Ser. No. 62/023,434, filed on 11 Jul. 2014, and incorporated by reference herein in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is Anbalagan_{—}PEDV. The text file is 115 KB; it was created on 10 Jul. 2015; and it is being submitted electronically via EFS-Web, concurrent with the filing of the specification.

### FIELD OF THE INVENTION

The present disclosure relates generally to vaccines and more specifically to an inactivated vaccine to prevent infection of pigs by porcine epidemic diarrhea virus (PEDV).

### BACKGROUND

Porcine epidemic diarrhea Virus (PEDV) is a severe and highly contagious swine disease. While older pigs have a chance of survival, 80 to 100 percent of the PEDV-infected piglets die within 24 hours of being infected. PEDV spreads primarily through fecal-oral contact (Pospischil et al., 2002; Song and Park, 2012). Once internalized it destroys the inner lining of piglets' intestines, making them incapable of digesting and deriving nutrition from milk and feed (Pospischil et al., 2002). The virus causes diarrhea, vomiting and death from severe dehydration and starvation in piglets. Moreover, the infected piglets shed virus for seven to ten days (Song and Park, 2012).

Porcine epidemic diarrhea virus (PEDV) circulated throughout Europe and Asia during the past three decades before being detected in swine in the United States in May 2013 (1-7). Since its introduction to the U.S., PEDV has been identified in 30 states by the National Animal Health Laboratory Network, as of May 2014. It is characterized by watery diarrhea, vomiting, dehydration, and high mortality rates in suckling pigs (8-10). The U.S. PEDV strains are phylogenetically subgroup IIa, which is similar to PEDV circulating in Asia in 2011 and 2012 (6, 7).

PEDV is a member of the Coronavirinae family and belongs to alphacoronavirus genera. These viruses are enveloped, positive-sense, single-stranded RNA and with a nucleocapsid of helical symmetry of 130nm in diameter (Pensaert and de Bouck, 1978; Spaan et al., 1988; Kocherhans et al., 2001). Their genomic size ranges from an approximately 26 to 32 Kb, relatively large for an RNA virus. Coronavirus are the largest viruses that are known to infect humans, other mammals, and birds, usually causing subclinical respiratory or gastrointestinal diseases. The PEDV subgenomic mRNAs, which are transcribed from the genome, produce viral protein subunits, such as the spike (S, ˜180-220 kDa), envelope (E, ˜8.8 kDa), membrane (M, 27-32 kDa), nucleoprotein (N, 55-58 kDa), and several other proteins of unknown function (Kocherhans et al., 2001; Li et al., 2012).

About two-thirds of the 5′ end of the genome encodes a replicase protein. These proteins are encoded by two slightly overlapping open reading frames (ORF), ORF1a and ORF1b (Bridgen et al., 1988; Kocherhans et al., 2001). These two ORF subunits are connected by a ribosomal frame shift site in all the coronaviruses. This regulates the ratio of the two polypeptides encoded by ORF1a and the read-through product ORF lab. About 70-80% of the translation products are terminated at the end of ORF1a, and the remaining 20-30% continues to transcribe until the end of ORF lb. The polypeptides are posttranslationally processed by viral encoded proteases (Bridgen et al., 1988; Park et al., 2012; Park et al., 2013). These proteases are encoded within ORF1a and the polymerase-/helicase-function are encoded by ORF1b. The analysis and amino acid alignment of N, M, E, ORF3 and S gene sequences of the highly virulent PEDV strain CV777 shows that PEDV occupies an intermediate position between the two well-characterized members of the group I corona viruses, TGEV and human coronavirus (HCoV-229E) (Pratelli 2011).

The nucleoprotein (N) subunit is a RNA-binding protein, and plays an important role in both virus RNA synthesis and modulating host cell processes. Phosphorylation and dephosphorylation may regulate these processes by exposing various functional motifs (Spencer et al., 2008; Hsieh et al., 2005). The N protein subunit has been implicated in various functions throughout the coronavirus life cycle including encapsulation, packaging, correct folding of the RNA molecule, the deregulation of the host cell cycle (Surjit, et at., 2006; Masters and Sturman, 1990), inhibition of interferon production, up-regulation of COX2 production, up-regulation of AP1 activity, induction of apoptosis, association with host cell proteins, and RNA chaperone activity (Stohlman et al., 1988; Tang et al., 2005; Nelson et al., 2000).

The PEDV E protein subunit is a homooligomer which interacts with the membrane (M) protein subunit in the budding compartment of the host cell, which is located between the endoplasmic reticulum (ER) and the Golgi complex (Duarte et al., 1994; Bridgen et al., 1998). The E protein subunit is a component of the viral envelope that plays a central role in virus morphogenesis and assembly. It also acts as a viroporin, inducing the formation of hydrophilic pores in cellular membranes and is sufficient to form virus-like particles (Madan et al., 2005). The PEDV E protein subunit has no effect on the intestinal epithelial cells (IEC) growth, cell cycle and cyclin-A expression. In contrast, the cells expressing PEDV E protein induce higher levels of IL-8 than control cells (Xu et al., 2013). Studies have shown that PEDV E protein induces ER-stress and activates transcription factor NF-κB, which is responsible for the up-regulation of interleukin 8 (IL-8) and Bc1-2 expression (Liao et al., 2006; Liao et al., 2004; Xu et al., 2013).

The M protein subunit of PEDV is the most abundant component of the viral envelope. In silico analysis of the M protein subunit shows that it consists of a triple-transmembrane segment flanked by a short amino-terminal domain on the exterior of the virion and a long carboxy-tail located inside the virion. The M protein subunit of coronaviruses is indispensable in the assembly process and budding of virions (Zhang et al., 2012). The immune reaction to the M protein of coronaviruses plays an important role in the induction of protection and in mediating the course of the disease (Zhang et al., 2012). Monoclonal antibodies against the M protein subunit of coronaviruses have virus-neutralizing activity in the presence of complement (Qian et al., 2006). Furthermore, the M protein subunit of coronavirus can also stimulate the production of alpha-interferon (α-IFN) which can inhibit viral replication (Xing et al., 2009).

The function of the PEDV ORF3 product subunit remains enigmatic, however computational modeling of PEDV OFR3 protein subunit shows that it may function as an ion channel and regulate virus production (Wang et al., 2012). Small interfering RNA (siRNA) knockdown of ORF3 gene in PEDV infected cells reduces the number of particles released from the cells (Wang et al., 2012). Passing PEDV in cell culture leads to the truncation or loss of ORF3 (Schmitz et al., 1998; Utiger et al., 1995). Homologues of the ORF3 protein subunit are found in all other alphacoronaviruses. The ORF3 protein of hCoV-NL63 was shown to be N-glycosylated at the amino terminus and incorporated into virions. However, deletion of the ORF3 gene from the viral genome had little effect on virus replication in vitro (Donaldson et al., 2008). Similar to other alphacoronaviruses (TGEV and, HCoV-229E) loss of PEDV ORF3 does not affect its replication in vitro (Dijkman et al., 2006; Woods, 2001). Despite a non-essential role in cell culture, the maintenance of the ORF3 gene in alphacoronavirus field strains strongly points to an important role of the ORF3 protein in natural infection in the animal host. Consistently, the loss of virulence of live-attenuated PEDV vaccine strains has been associated with mutations in the ORF3 gene resulting from cell culture adaptation (Song et al., 2007). However, this loss of virulence can also be attributed to concomitant mutations in other genes such as the spike protein gene (Park et al., 2008; Sato et al., 2012). The specific function of the ORF3 protein (and other viral proteins in the 3′ genome region) in PEDV replication and pathogenesis can now be investigated using the reverse genetics system (Li et al., 2013).

The spike protein of the PEDV is a large glycoprotein of ˜180 to 200 kDa, and belongs to the class I fusion proteins (Bosch et al., 2003). The functional S protein subunit forms a homotrimer on the virion surface. The coronavirus S proteins consists of two subunits and are cleaved by host proteases into the N-terminal S1 subunit and the C-terminal membrane-anchored S2 subunit. The S1 subunit binds to its receptor on the host cell, while the S2 subunit is responsible for fusion activity (Park et al., 2007; de Haan et al., 2004). This cleavage initiates the cell-to-cell fusion and virus entry into cells (Spaan et al., 2008; Simmons et al., 2004). Various proteases are known to be utilized for cleavage of the S protein subunit of each coronavirus. For example, in murine coranavirus mouse hepatitis virus (MHV), the basic amino acid cluster in the middle of the S protein is cleaved by a protease, furin, during its biogenesis. The cleaved S protein subunit is retained on the virion and infected-cell surfaces, inducing cell-to-cell fusion (Spaan et al., 2008). In contrast, S proteins of severe acute respiratory syndrome coronavirus (SARS-CoV), nonfusogenic MHV-2, and HCoV-229E, have no furin recognition site, therefore these S proteins are not cleaved during their biogenesis (Simmons et al., 2004; Matsuyama et al., 2004; Yoshikura et al., 1988; Shirato et al., 2011). These S proteins without a furin recognition site are cleaved by endosomal proteases, such as cathepsins, and other proteases activated by the low-pH environment (Shirato et al., 2011). These coronaviruses, once bound to the receptor, are transported to the endosome, where the S protein subunit is cleaved and activated for fusion, which, in turn, results in the release of the virus genome into the cytoplasm from the endosome (Shirato et al., 2011). Thus, these coronavirus fail to induce syncytia in infected cells, and the S protein on the virion is not in a cleaved form (Shirato et al., 2011). Furthermore, the efficiency of infection of these coronavirus is not influenced by exogenous proteases. Similarly, PEDV has uncleaved S protein and PEDV-infected cells produce syncytia only after treatment with an exogenous protease, features similar to those of the coronavirus described above (Duarte et al., 1994; Durante and Laude, 1994). However, without the exogenous protease treatment, PEDV cannot grow efficiently in vitro (Park et al., 2007; Shirato et al., 2011). This explains the need for protease mediated cleavage of PEDV S protein subunit for virulence and in vitro propagation.

The complete genomic sequences of PEDV isolated from outbreaks in Minnesota and Iowa are available in the GenBank (Colorado, USA: USA/Colorado/2013, accession no. KF272920; 13-019349, accession no. KF267450 and ISU13-19338E-IN-homogenate, accession number KF650370). The genetic and phylogenetic analysis of the three U.S. strains reveals a close relationship with Chinese PEDV strains and possible Chinese origin. The U.S. PEDV strains underwent evolutionary divergence, and are classified into two sublineages. The three emergent U.S. strains are most closely related to a strain isolated in 2012 from Anhui Province in China, which might be the result of multiple recombination events between different genetic lineages or sublineages of PEDV. Molecular clock analysis of the PEDV strain-divergence based on the complete genomic sequences shows an approximately 2 to 3 years' time-frame between the Chinese (December 2010) and the U.S. (May 2013) outbreaks [US-USDA, Technical note, PED. Fort Collins (Colo.): USDA; 2013]. The finding that the emergent U.S. PEDV strains share unique genetic features at the 5′-untranslated region with a bat coronavirus provided further support of the evolutionary origin of PEDV from bats and potential cross-species transmission (Graham and Baric 2010; Wang et al., 2014).

Modified-live vaccines (MLVs) have long been used in Asia for the control of PEDV (11-13). The strain 83P-5, attenuated by one-hundred cell culture passages, has been licensed in Japan as an attenuated live PEDV vaccine (13). During the attenuation process, this strain acquired fourteen amino acid changes in the immunodominant S protein, which is critical for virus binding to cell receptors and is the target of neutralizing antibodies (14-19). The live attenuated DR13 vaccine strain of PEDV had thirteen of these fourteen mutations as well (13). Serial passage of 83P-5 in Vero cells resulted in attenuation of virulence in vivo and the strong selection for the viral S gene was associated with these phenotypic changes.

Classically attenuated cell culture passaged PEDV also shows mutations in open reading frame 3 (ORF3) and changes to restriction fragment length polymorphism (RFLP) cut patterns, which have been used to distinguish MLV from field strains (10,20). In vivo, high-passage (x>100) MLVs were attenuated in sows and piglets while still capable of inducing a robust immune response (20). While attenuated in their ability to cause disease, the safety of using MLV has been questioned, as MLV are shed in the environment. Virus was detected in feces of 3-day old piglets up to seven days after oral inoculation with DR13 passage 100 (12, 21). In 2010, PEDV was isolated from diarrheic pigs in China that had a close phylogenetic relationship to two MLV vaccines, suggesting it may have evolved from a MLV (22).

While modified live vaccines may elicit a more robust and protective immune response than inactivated virus vaccines (13), efficacy is often lacking (23). In late 2010, China experienced a severe outbreak of PEDV in suckling pigs, causing drastic economic losses (24). This outbreak was caused by a strain with a phylogenetically distinct S gene from other Chinese strains and from vaccine strain CV777 (24). In 2012, the PEDV infection rates in vaccinated herds in China increased dramatically. Phylogenetic analysis of new variants from the outbreak showed insertions and deletions in antigenic regions of the S gene that may have influenced the efficacy of the CV777 MLV (25). Investigation into whether an inactivated vaccine can elicit a protective immune response could lead to the development of vaccines more closely related to field strains and avoid potential antigenic changes due to excessive in vitro cultivation.

There is currently one PEDV vaccine in the U.S. for use in sows (Harris Vaccines, SirraVax RNA platform technology). With mortality rates as high as 100% in suckling piglets and total losses estimated over 5 million animals in the U.S. in less than one year, PEDV vaccines are critically needed. The U.S. Department of Agriculture allows for the production of autogenous vaccines to address emerging diseases however the difficulty in propagating PEDV in cell culture increases the difficulty in producing efficacious inactivated vaccines. Here, PEDV was isolated from pooled intestinal homogenate and passaged in cell culture. Inactivated cell culture derived viral vaccines were immunogenic when administered to naïve pigs. To our knowledge, this is the first demonstration of immunogenicity of an inactivated U.S. PEDV vaccine trial in pigs in the U.S.

### SUMMARY OF THE INVENTION

In one aspect, the invention is a nucleotide sequence of SEQ ID NO. 1. The nucleotide sequence may include, for example, the S1 and S2 domains of the S protein gene (i.e., spike or S domain) of porcine epidemic diarrhea virus. In another embodiment, the nucleotide sequence may further include the nucleoprotein (N) region of the N subunit gene of porcine epidemic diarrhea virus. In yet another embodiment, the nucleotide sequence may further include the E region of the E subunit gene of porcine epidemic diarrhea virus. In yet another embodiment, the nucleotide sequence may further include the M region of the M subunit gene of porcine epidemic diarrhea virus. In yet another embodiment, the nucleotide sequence may further include the ORF regions of the ORF subunit genes of porcine epidemic diarrhea virus.

In another aspect, the invention is a composition or vaccine comprising SEQ ID NO. 1. The composition or vaccine may include, for example, the S1 and S2 domains of the S protein gene of porcine epidemic diarrhea virus. In another embodiment, the composition or vaccine may further include the nucleoprotein (N) region of the N subunit gene of porcine epidemic diarrhea virus. In yet another embodiment, the composition or vaccine may further include the E region of the E subunit gene of porcine epidemic diarrhea virus. In yet another embodiment, the composition or vaccine may further include the M region of the M subunit gene of porcine epidemic diarrhea virus. In yet another embodiment, the composition or vaccine may further include the ORF regions of the ORF subunit genes of porcine epidemic diarrhea virus.

In another aspect, the invention is a vaccine or composition comprising SEQ ID NO. 1 and one or more pharmaceutically or veterinarily acceptable carriers, adjuvants, vehicles or excipients. The vaccine may include, for example, the S1 and S2 domains of the S protein gene of porcine epidemic diarrhea virus. In another embodiment, the composition or vaccine may further include the nucleoprotein (N) region of the N subunit gene of porcine epidemic diarrhea virus. In yet another embodiment, the composition or vaccine may further include the E region of the E subunit gene of porcine epidemic diarrhea virus. In yet another embodiment, the composition or vaccine may further include the M region of the M subunit gene of porcine epidemic diarrhea virus. In yet another embodiment, the composition or vaccine may further include the ORF regions of the ORF subunit genes of porcine epidemic diarrhea virus. In another embodiment, the composition or vaccine may include one or more other antigens.

In yet another aspect, the invention is a method of vaccinating a host susceptible to porcine epidemic diarrhea virus comprising at least one administration of a composition or vaccine comprising a virus encoded by SEQ ID NO. 1 and one or more pharmaceutically or veterinarily acceptable carriers, adjuvants, vehicles or excipients. In one or more embodiments, the immunoprotective vaccine is as described above. In another embodiment, the method of vaccinating may include one or more other antigens.

In another aspect, the invention is a composition or vaccine comprising a protein encoded by SEQ ID NO. 10. In an embodiment, the composition or vaccine including the protein encoded by SEQ ID NO. 10 is inactivated. In another embodiment, the composition or vaccine includes one or more pharmaceutically or veterinarily acceptable carriers, adjuvants, vehicles or excipients.

In yet another aspect, the invention is a method of vaccinating a host susceptible to porcine epidemic diarrhea virus comprising at least one administration of a composition or vaccine that includes the protein encoded by SEQ ID NO. 10.

### BRIEF DESCRIPTION OF THE DRAWINGS

A full and enabling description of the present invention is set forth in the remainder of the specification, including reference to the accompanying figures, wherein:

FIG. 1 shows a phylogenetic analysis of 12 full length porcine epidemic diarrhea virus genomes.

FIG. 2A shows a partial view of the full length nucleotide sequence of NPL PEDV 2013 P10.1 (SEQ ID NO. 1).

FIG. 2B shows a partial view of the full length nucleotide sequence of NPL PEDV 2013 P 10.1 (SEQ ID NO. 1).

FIG. 2C shows a partial view of the full length nucleotide sequence of NPL PEDV 2013 P 10.1 (SEQ ID NO. 1).

FIG. 2D shows a partial view of the full length nucleotide sequence of NPL PEDV 2013 P10.1 (SEQ ID NO. 1).

FIG. 2E shows a partial view of the full length nucleotide sequence of NPL PEDV 2013 P10.1 (SEQ ID NO. 1).

FIG. 2F shows a partial view of the full length nucleotide sequence of NPL PEDV 2013 P10.1 (SEQ ID NO. 1).

FIG. 2G shows a partial view of the full length nucleotide sequence of NPL PEDV 2013 P10.1 (SEQ ID NO. 1).

FIG. 2H shows a partial view of the full length nucleotide sequence of NPL PEDV 2013 P10.1 (SEQ ID NO. 1).

FIG. 3 shows the amino acid sequence for the NPL PEDV 2013 P10.1 Envelope protein.

FIG. 4 shows the amino acid sequence for the NPL PEDV 2013 P10.1 Membrane protein.

FIG. 5 shows the amino acid sequence for the NPL PEDV 2013 P10.1 Nucleocapsid protein.

FIG. 6A shows a partial view of the amino acid sequence for the NPL PEDV 2013 P10.1 ORFlab protein (SEQ ID NO. 8).

FIG. 6B shows a partial view of the amino acid sequence for the NPL PEDV 2013 P10.1 ORFlab protein (SEQ ID NO. 8).

FIG. 6C shows a partial view of the amino acid sequence for the NPL PEDV 2013 P10.1 ORFlab protein (SEQ ID NO. 8).

FIG. 7 shows the amino acid sequence for the NPL PEDV 2013 P10.1 ORF 3 protein.

FIG. 8A shows a partial view of the amino acid sequence for the NPL PEDV 2013 P10.1 Spike protein (SEQ ID NO. 10).

FIG. 8B shows a partial view of the amino acid sequence for the NPL PEDV 2013 P10.1 Spike protein (SEQ ID NO. 10).

### DETAILED DESCRIPTION OF THE INVENTION

Unless otherwise explained, all technical and scientific terms used herein have the same meaning as commonly understood by one of ordinary skill in the art to which this disclosure belongs. The singular terms “a”, “an”, and “the” include plural referents unless context clearly indicates otherwise. Similarly, the word “or” is intended to include “and” unless the context clearly indicate otherwise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Table 1 lists the sequences utilized in the invention.

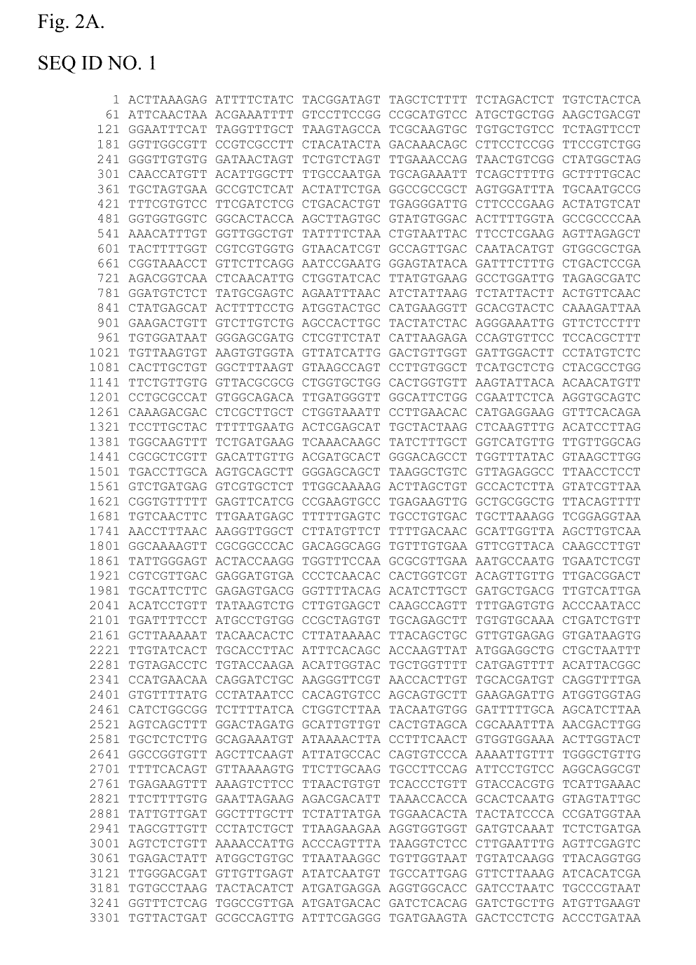

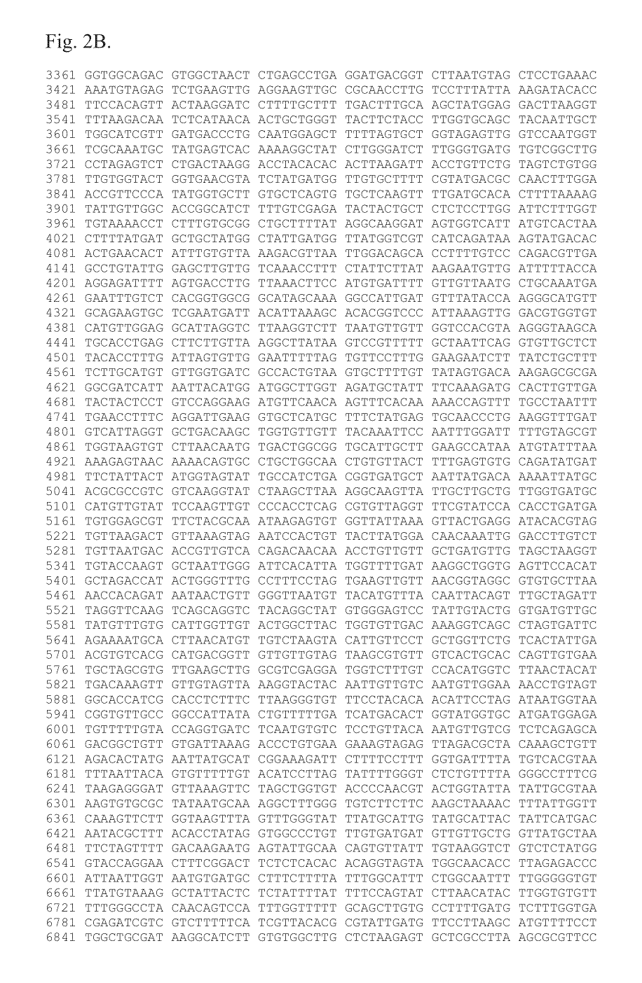

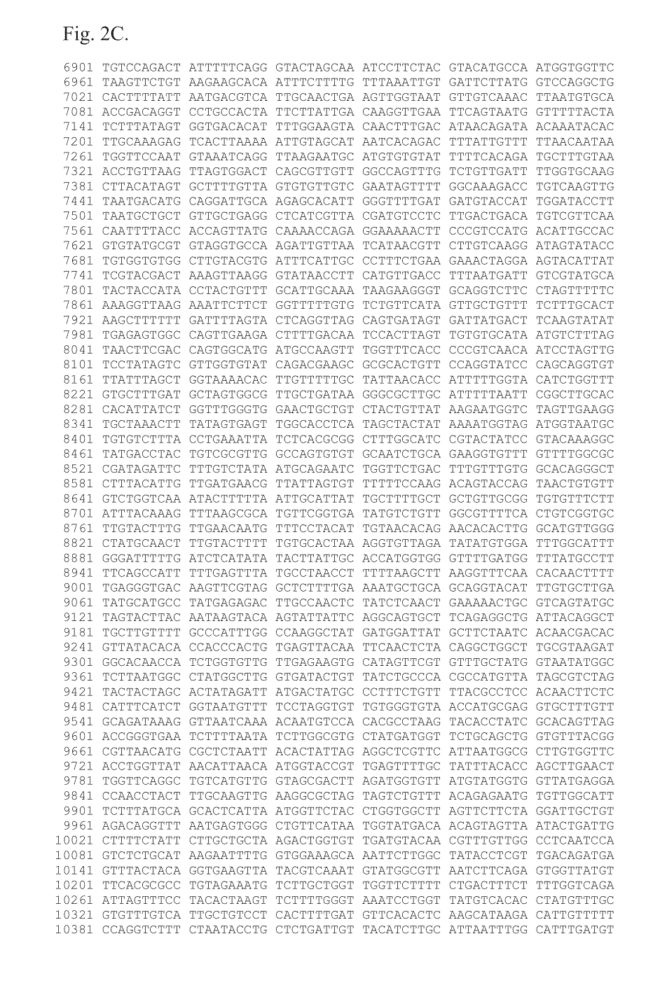

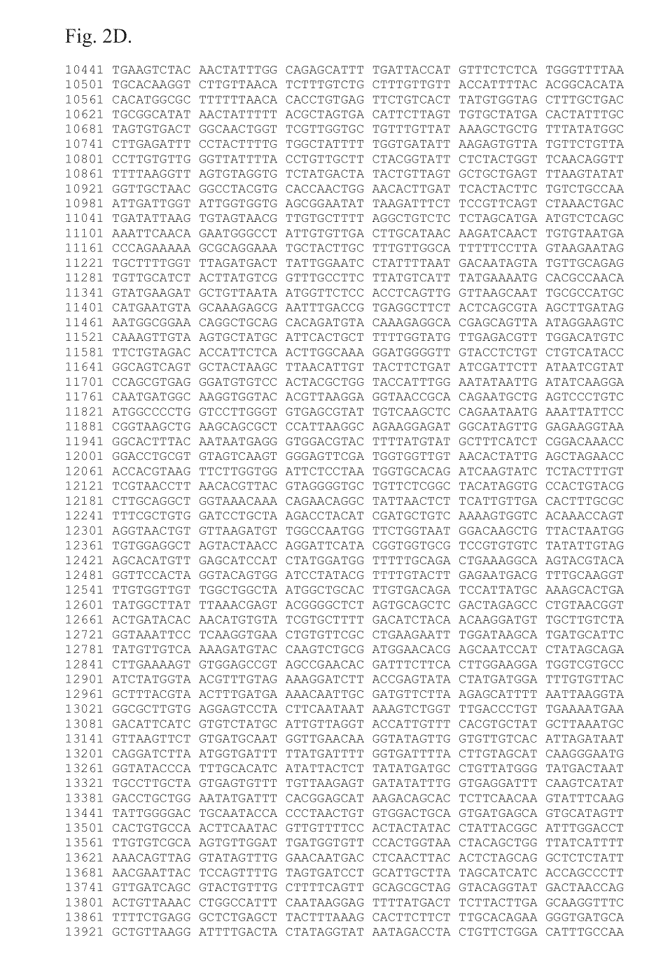

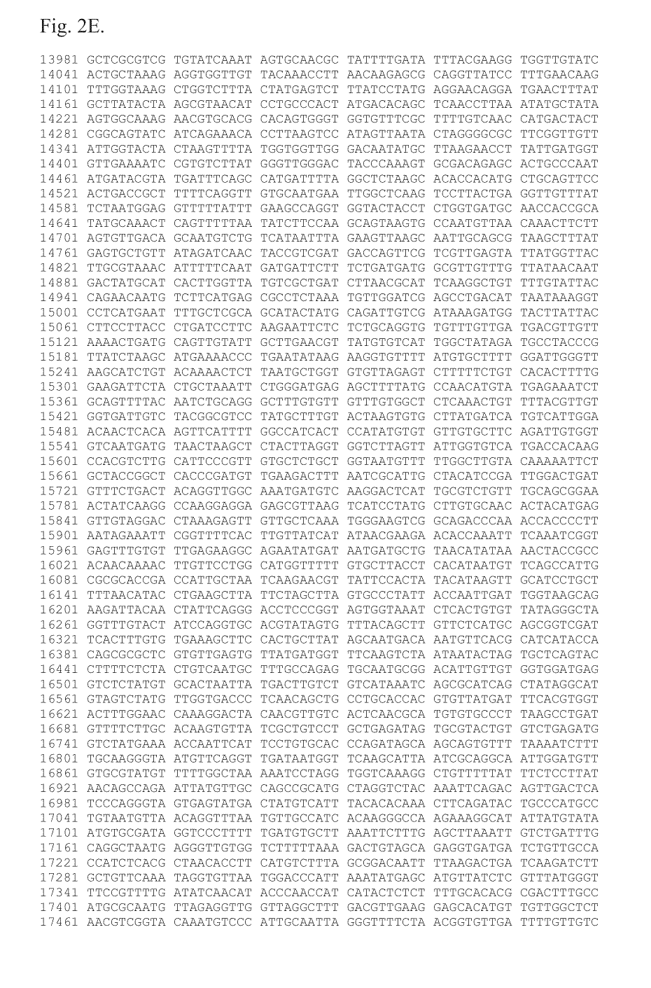

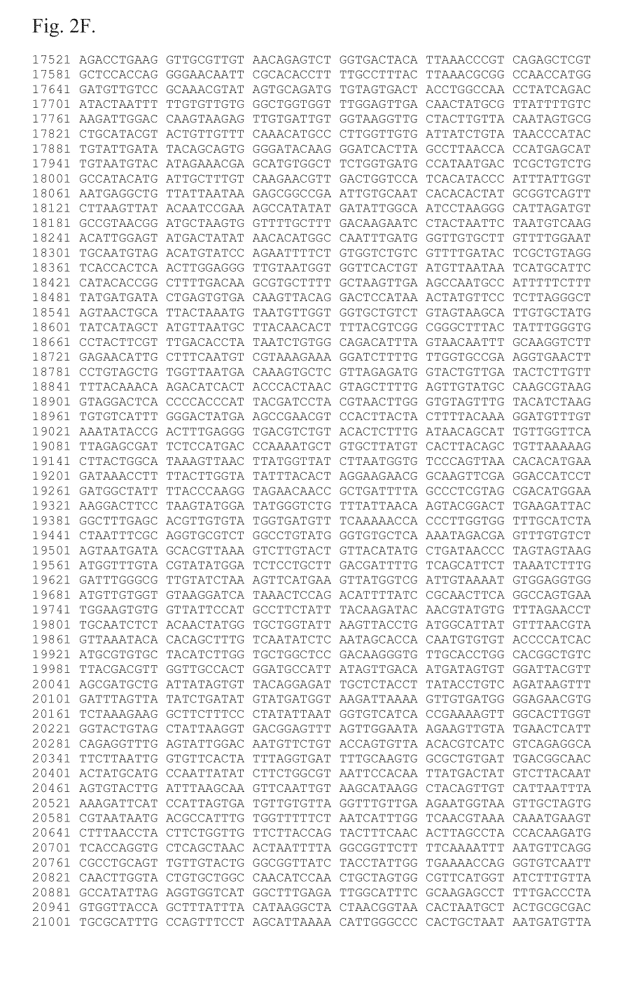

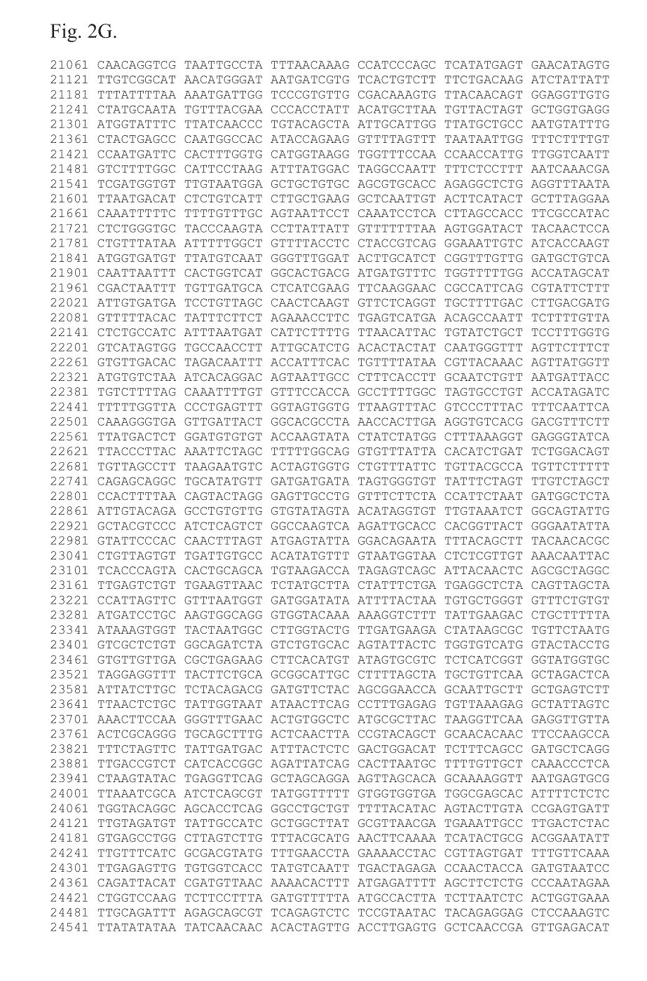

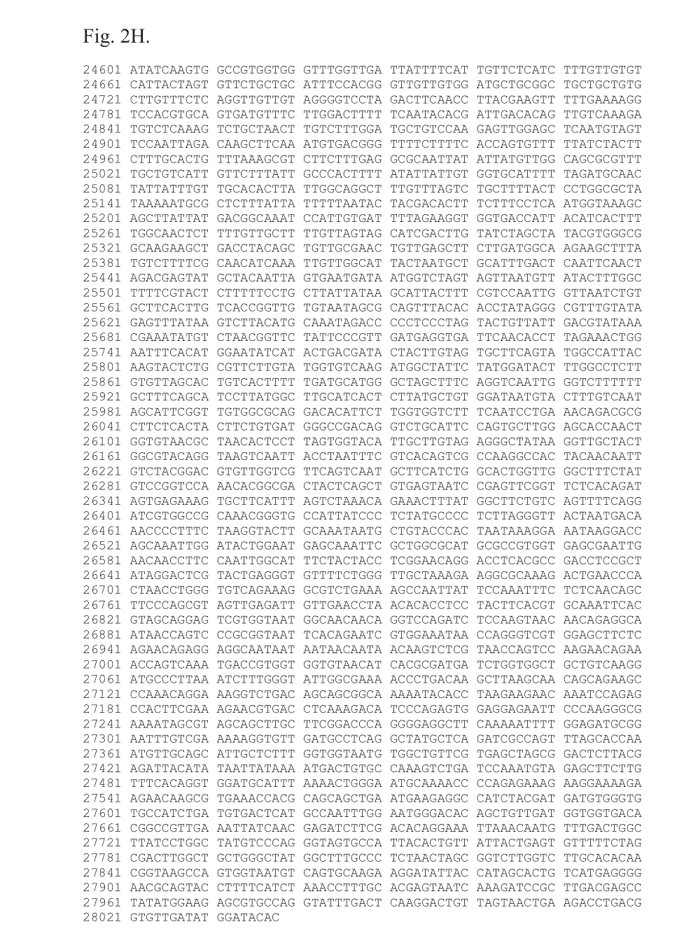

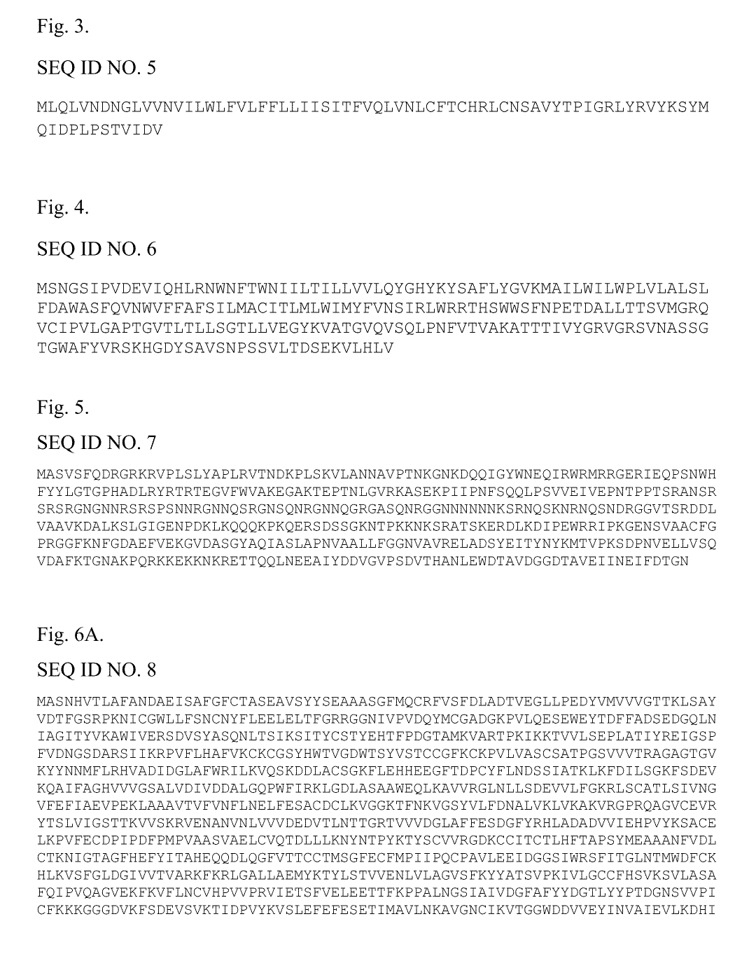

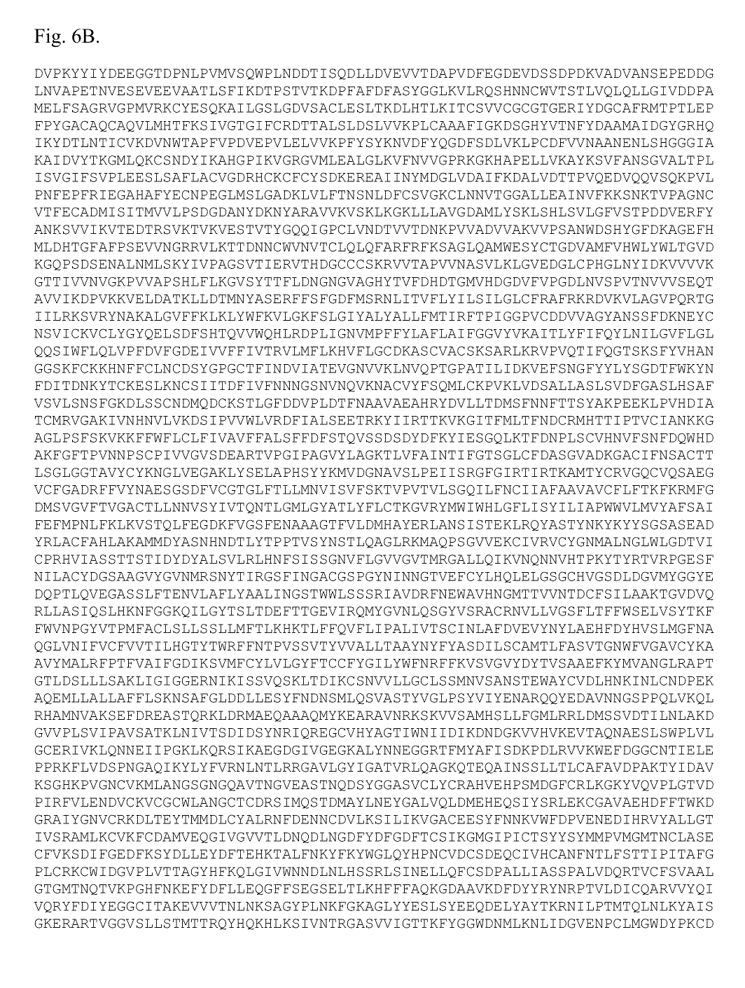

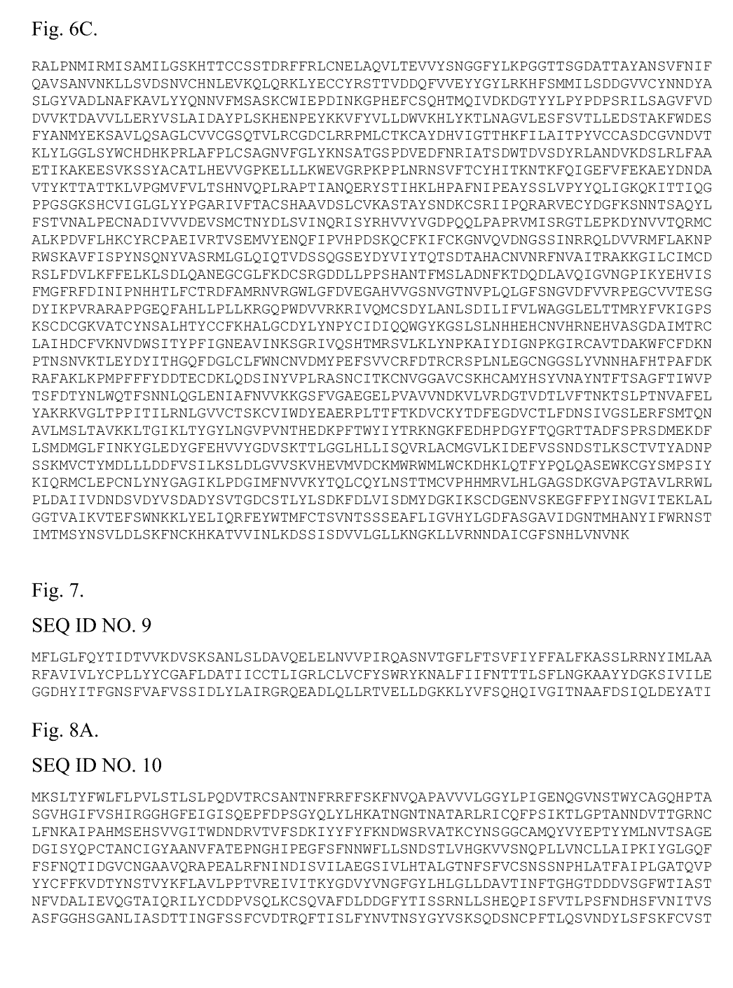

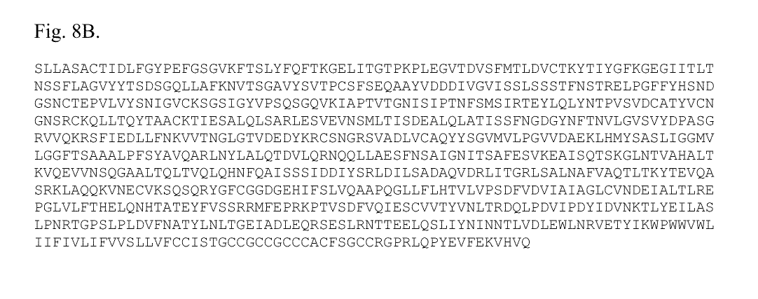

TABLE 1

| SEQ ID NO. | TYPE | Description |
|---|---|---|
| 1 | DNA | Nucleotide sequence of NPL PEDV 2013 P10.1 |
| | | (FIG. 2) |
| 2 | DNA | rt-RT-PCR forward PEDV primer 5′ → 3′ |
| 3 | DNA | rt-RT-PCR reverse PEDV primer 3′ → 5′ |
| 4 | DNA | PEDV probe 5′ → 3′ |
| 5 | protein | NPL PEDV 2013 P10.1 Envelope protein |
| 6 | protein | NPL PEDV 2013 P10.1 Membrane protein |
| 7 | protein | NPL PEDV 2013 P10.1 Nucleocapsid protein |
| 8 | protein | NPL PEDV 2013 P10.1 ORF1ab protein |
| 9 | protein | NPL PEDV 2013 P10.1 ORF 3 protein |
| 10 | protein | NPL PEDV 2013 P10.1 Spike protein |

The nucleotide sequence of the invention encodes antigens or immunogens capable of protecting against porcine epidemic diarrhea virus (PEDV). That is, it is capable of stimulating an immune response in an animal. By “antigen” or “immunogen” means a substance that induces a specific immune response in a host animal. The antigen of the instant invention is a nucleotide sequence or portion thereof of an organism; a piece or fragment of DNA capable of inducing an immune response upon presentation to a host animal; a polypeptide, an epitope, a hapten, an inactivated viral culture or any combination thereof.

The term “immunogenic protein, polypeptide, or peptide” as used herein includes polypeptides that are immunologically active in the sense that once administered to the host, it is able to evoke an immune response of the humoral and/or cellular type directed against the protein. A protein fragment according to the invention has at least one epitope or antigenic determinant. An “immunogenic” protein or polypeptide, as used herein, includes the full-length sequence of the protein, analogs thereof, or immunogenic fragments thereof.

The invention encompasses fragments and variants of the antigenic polypeptide. Thus, the term “immunogenic protein, polypeptide, or peptide” further contemplates deletions, additions and substitutions to the sequence, so long as the polypeptide functions to produce an immunological response as defined herein. The term “conservative variation” denotes the replacement of an amino acid residue by another biologically similar residue, or the replacement of a nucleotide in a nucleic acid sequence such that the encoded amino acid residue does not change or is another biologically similar residue. In this regard, particularly preferred substitutions will generally be conservative in nature, i.e., those substitutions that take place within a family of amino acids. For example, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine, threonine, tyrosine. Phenylalanine, tryptophan, and tyrosine are sometimes classified as aromatic amino acids. Examples of conservative variations include the substitution of one hydrophobic residue such as isoleucine, valine, leucine or methionine for another hydrophobic residue, or the substitution of one polar residue for another polar residue, such as the substitution of arginine for lysine, glutamic acid for aspartic acid, or glutamine for asparagine, and the like; or a similar conservative replacement of an amino acid with a structurally related amino acid that will not have a major effect on the biological activity. Proteins having substantially the same amino acid sequence as the reference molecule but possessing minor amino acid substitutions that do not substantially affect the immunogenicity of the protein are, therefore, within the definition of the reference polypeptide. All of the polypeptides produced by these modifications are included herein. The term “conservative variation” also includes the use of a substituted amino acid in place of an unsubstituted parent amino acid provided that antibodies raised to the substituted polypeptide also immunoreact with the unsubstituted polypeptide.

The term “epitope” refers to the site on an antigen or hapten to which specific B cells and/or T cells respond. The term is also used interchangeably with “antigenic determinant” or “antigenic determinant site”. Antibodies that recognize the same epitope can be identified in a simple immunoassay showing the ability of one antibody to block the binding of another antibody to a target antigen.

An “immunological response” to a composition or vaccine is the development in the host of a cellular and/or antibody-mediated immune response to a composition or vaccine of interest. Usually, an “immunological response” includes but is not limited to one or more of the following effects: the production of antibodies, B cells, helper T cells, and/or cytotoxic T cells, directed specifically to an antigen or antigens included in the composition or vaccine of interest. Preferably, the host will display either a therapeutic or protective immunological response such that resistance to new infection will be enhanced and/or the clinical severity of the disease reduced. Such protection will be demonstrated by either a reduction or lack of symptoms normally displayed by an infected host, a quicker recovery time and/or a lowered viral titer in the infected host.

Synthetic antigens are also included within the definition, for example, polyepitopes, flanking epitopes, and other recombinant or synthetically derived antigens. See, e.g., Bergmann et al., 1993; Bergmann et al., 1996; Suhrbier, 1997; Gardner et al., 1998. Immunogenic fragments, for purposes of the present invention, will usually include at least about 3 amino acids, at least about 5 amino acids, at least about 10-15 amino acids, or about 15-25 amino acids or more amino acids, of the molecule. There is no critical upper limit to the length of the fragment, which could comprise nearly the full-length of the protein sequence, or even a fusion protein comprising at least one epitope of the protein.

Accordingly, a minimum structure of a polynucleotide expressing an epitope is that it has nucleotides encoding an epitope or antigenic determinant of a PEDV polypeptide. A polynucleotide encoding a fragment of a PEDV polypeptide may have a minimum of 15 nucleotides, about 30-45 nucleotides, about 45-75, or at least 57, 87 or 150 consecutive or contiguous nucleotides of the sequence encoding the polypeptide. Epitope determination procedures, such as, generating overlapping peptide libraries (Hemmer et al., 1998), Pepscan (Geysen et al., 1984; Geysen et al., 1985; Van der Zee R. et al., 1989; Geysen, 1990; Multipin. RTM. Peptide Synthesis Kits de Chiron) and algorithms (De Groot et al., 1999; PCT/US2004/022605) can be used in the practice of the invention.

The term “nucleic acid” or “nucleotide” refers to RNA or DNA that is linear or branched, single or double stranded, or a hybrid thereof. The term also encompasses RNA/DNA hybrids. The following are non-limiting examples of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes and primers. A polynucleotide may comprise modified nucleotides, such as methylated nucleotides and nucleotide analogs, uracyl, other sugars and linking groups such as fluororibose and thiolate, and nucleotide branches. The sequence of nucleotides may be further modified after polymerization, such as by conjugation, with a labeling component. Other types of modifications included in this definition are caps, substitution of one or more of the naturally occurring nucleotides with an analog, and introduction of means for attaching the polynucleotide to proteins, metal ions, labeling components, other polynucleotides or solid support. The polynucleotides can be obtained by chemical synthesis or derived from a microorganism.

The term “gene” is used broadly to refer to any segment of polynucleotide associated with a biological function. Thus, genes include introns and exons as in genomic sequence, or just the coding sequences as in cDNAs and/or the regulatory sequences required for their expression. For example, gene also refers to a nucleic acid fragment that expresses mRNA or functional RNA, or encodes a specific protein, and which includes regulatory sequences.

The invention further comprises a complementary strand to a polynucleotide encoding a PEDV antigen, epitope or immunogen. The complementary strand can be polymeric and of any length, and can contain deoxyribonucleotides, ribonucleotides, and analogs in any combination.

The terms “protein”, “peptide”, “polypeptide” and “polypeptide fragment” are used interchangeably herein to refer to polymers of amino acid residues of any length. The polymer can be linear or branched, it may comprise modified amino acids or amino acid analogs, and it may be interrupted by chemical moieties other than amino acids. The terms also encompass an amino acid polymer that has been modified naturally or by intervention; for example disulfide bond formation, glycosylation, lipidation, acetylation, phosphorylation, or any other manipulation or modification, such as conjugation with a labeling or bioactive component.

An “isolated” biological component (such as a nucleic acid or protein or organelle) refers to a component that has been substantially separated or purified away from other biological components in the cell of the organism in which the component naturally occurs, for instance, other chromosomal and extra-chromosomal DNA and RNA, proteins, and organelles. Nucleic acids and proteins that have been “isolated” include nucleic acids and proteins purified by standard purification methods. The term also embraces nucleic acids and proteins prepared by recombinant technology as well as chemical synthesis.

The term “purified” as used herein does not require absolute purity; rather, it is intended as a relative term. Thus, for example, a purified polypeptide preparation is one in which the polypeptide is more enriched than the polypeptide is in its natural environment. That is the polypeptide is separated from cellular components. By “substantially purified” it is intended that such that the polypeptide represents several embodiments at least 60%, at least 70%, at least 80%, at least 90%, at least 95%, or at least 98%, or more of the cellular components or materials have been removed. Likewise, the polypeptide may be partially purified. By “partially purified” is intended that less than 60% of the cellular components or material is removed. The same applies to polynucleotides. The polypeptides disclosed herein can be purified by any of the means known in the art.

Fragments and variants of the disclosed polynucleotides and polypeptides encoded thereby are also encompassed by the present invention. By “fragment” is intended a portion of the polynucleotide or a portion of the antigenic amino acid sequence encoded thereby. Fragments of a polynucleotide may encode protein fragments that retain the biological activity of the native protein and hence have immunogenic activity as noted elsewhere herein. Fragments of the polypeptide sequence retain the ability to induce a protective immune response in an animal.

“Variants” is intended to mean substantially similar sequences. For polynucleotides, a variant comprises a deletion and/or addition of one or more nucleotides at one or more sites within the native polynucleotide and/or a substitution of one or more nucleotides at one or more sites in the native polynucleotide. As used herein, a “native” polynucleotide or polypeptide comprises a naturally occurring nucleotide sequence or amino acid sequence, respectively. Variants of a particular polynucleotide of the invention (i.e., the reference polynucleotide) can also be evaluated by comparison of the percent sequence identity between the polypeptide encoded by a variant polynucleotide and the polypeptide encoded by the reference polynucleotide. “Variant” protein is intended to mean a protein derived from the native protein by deletion or addition of one or more amino acids at one or more sites in the native protein and/or substitution of one or more amino acids at one or more sites in the native protein. Variant proteins encompassed by the present invention are biologically active, that is they the ability to elicit an immune response.

As used herein, the term “derivative” or “variant” refers to a polypeptide, or a nucleic acid encoding a polypeptide, that has one or more conservative amino acid variations or other minor modifications such that (1) the corresponding polypeptide has substantially equivalent function when compared to the wild type polypeptide or (2) an antibody raised against the polypeptide is immunoreactive with the wild-type polypeptide. These variants or derivatives include polypeptides having minor modifications of the NPL-PEDV polypeptide primary amino acid sequences that may result in peptides which have substantially equivalent activity as compared to the unmodified counterpart polypeptide. Such modifications may be deliberate, as by site-directed mutagenesis, or may be spontaneous. The term “variant” further contemplates deletions, additions and substitutions to the sequence, so long as the polypeptide functions to produce an immunological response as defined herein.

The term “conservative variation” denotes the replacement of an amino acid residue by another biologically similar residue, or the replacement of a nucleotide in a nucleic acid sequence such that the encoded amino acid residue does not change or is another biologically similar residue. In this regard, particularly preferred substitutions will generally be conservative in nature, as described above.

The polynucleotides of the disclosure include sequences that are degenerate as a result of the genetic code, e.g., optimized codon usage for a specific host. As used herein, “optimized” refers to a polynucleotide that is genetically engineered to increase its expression in a given species. To provide optimized polynucleotides coding for PEDV polypeptides, the DNA sequence of the PEDV gene can be modified to 1) comprise codons preferred by highly expressed genes in a particular species; 2) comprise an A+T or G+C content in nucleotide base composition to that substantially found in said species; 3) form an initiation sequence of said species; or 4) eliminate sequences that cause destabilization, inappropriate polyadenylation, degradation and termination of RNA, or that form secondary structure hairpins or RNA splice sites. Increased expression of PEDV protein in said species can be achieved by utilizing the distribution frequency of codon usage in eukaryotes and prokaryotes, or in a particular species. The term “frequency of preferred codon usage” refers to the preference exhibited by a specific host cell in usage of nucleotide codons to specify a given amino acid. There are 20 natural amino acids, most of which are specified by more than one codon. Therefore, all degenerate nucleotide sequences are included in the disclosure as long as the amino acid sequence of the PEDV polypeptide encoded by the nucleotide sequence is functionally unchanged.

The present invention relates to porcine vaccines or pharmaceutical or immunological compositions which may comprise an effective amount of inactivated PEDV antigens and a pharmaceutically or veterinarily acceptable carrier, excipient, or vehicle.

The subject matter described herein is directed in part, to compositions and methods related to the inactivated PEDV antigen prepared in Vero and MARC-145 cells that was highly immunogenic and protected animals against challenge from PEDV strains.

### Virus Isolation

In May 2013, intestines from pigs in Iowa experiencing PEDV-like symptoms were submitted to Newport Laboratories for diagnostic testing. Intestines were homogenized in phosphate buffered saline and debris was removed by centrifugation at 10,000×g for 10 minutes followed by filtration through a 0.2 μm filter. Virus isolation was performed on Vero (ATCC CCL-81), Vero 76 (ATCC CRL-1586), and MARC-145 cells (26). All cells were maintained in Dulbecco's modification of Eagles medium (DMEM) with five percent fetal bovine serum and one percent L-glutamine. Confluent monolayers were washed three times with DMEM without serum prior to inoculation. For the initial infection of cells in 12-well plates, 200 μL of inoculum was adsorbed at 37° C. with +5% CO₂ for 1-2 hours with small amount of viral growth media (DMEM with 0.75 μg/mL TPCK-treated trypsin, and Normocin antibiotic (Invivogen)). The inoculum was rinsed from the plates with viral growth media and the cells were refed with viral growth media. Plates were incubated up to 5 days before being frozen, thawed, and passaged. Subsequent passages were performed by inoculating 200 μL of cell culture harvest onto confluent monolayers in 12-well plates. Viral replication was verified by real time reverse transcription PCR (rt-RT-PCR) (below) and indirect immunofluorescence (IFA). Viral cultures were scaled up in M145 25cm² flasks and 1700 cm² roller bottles.

### Indirect Immunofluorescence

IFA was performed on Vero or M145 96-well monolayers. Infected wells were fixed in cold ethyl alcohol and polyclonal rabbit anti-PEDV nucleoprotein antiserum (South Dakota State University) was added at 1:500. Cells were rinsed and then incubated with FITC labeled goat anti-rabbit IgG (Jackson Immunoresearch) at a dilution of 1:50, and then read using a fluorescent microscope. Tissue culture infective dose/mL (TCID₅₀/mL) was calculated using the Spearman-Karber method.

### Molecular Analysis

Viral RNA was extracted by using the MagMAX-96 viral RNA isolation kit (Life Technologies) according to the manufacturer's instructions. rt-RT-PCR was performed by using QIAGEN Quantitect RT-PCR with the PEDV primers and probe. For analytical purposes, negative samples were assigned a Ct value of 37.1, which corresponds to the detection limit of the method (approximately −1.0 TCID₅₀/mL). Method specificity was assessed by using various porcine enteric viruses, including transmissible gastroenteritis virus, group A rotavirus and porcine enterovirus, and no cross-reaction was observed. A standard curve was generated by serial dilution of M145 cell harvests containing 5.7 log₁₀ TCID₅₀/mL of PEDV, as determined by titration on M145 cells. rt-RT-PCR was performed with the following primers and probe: PEDV forward (SEQ ID NO. 2): 5′-ACG TCC GTA ACA CCT TCA AG-3′, PEDV reverse (SEQ ID NO. 3): 5′-GCT AGT GCC TGT ACC ATA GAT C-3′, and PEDV Probe (SEQ ID NO. 4): 5′-/5HEX/CGT GCC AGT AAT CAA CTC ACC CTT TGT/3IABkFQ/-3′.

### RNA Isolation for Next Generation Sequencing

M145 cells that showed 100% CPE following virus infection were used for RNA extraction. 20 ml of cell culture supernatant was filtered using the 0.2 μm bottle top filters (Thermo Scientific, Lenexa, Kans.). The filtrate was centrifuged at 50,000×g for 2 hours. Supernatant was discarded and the pellet was suspended in 1000 μl of water. Samples were concentrated to a final 100 μl volume using Amicon ultra centrifugal filters (0.5 ml; 50 KDa) (Millipore, Tullagreen, Ireland). Cellular DNA and RNA were removed by incubation with DNase I (25 units) (New England Biolabs, NEB, Ipswich, Mass.) and RNase A (25 units) (Qiagen, Valencia, Calif.) at 37° C. for 1 hour. RNA was extracted using Trizol LS Reagent (Life Technologies, Grand Island, N.Y.) according to manufacturer's instructions. The pellet containing RNA was resuspended in 20 μl of sterile H₂O.

### Sequencing and Data Analysis

Ten μg of total RNA was depleted of ribosomal RNA using GeneRead rRNA depletion kit (Qiagen) and RNA sequencing libraries were generated using the Ion Total RNA-seq kit v2 (Ion Torrent, Life Technologies) according to manufacturer's instructions. Sequencing was carried out using Ion Personal Genome Machine (PGM) sequencing platform (Life Technologies, Grand Island, N.Y.) as previously described (27). Sequence reads were assembled into contigs using the SeqMan NGen program (DNAstar, Madison, Wis.). Gaps in the sequence were filled by Sanger sequencing. Phylogenetic analysis on full genome sequences was performed using MEGA 6.0 software using Maximum Likelihood analysis with 1000 bootstrap replicates to verify tree topology. Sequence alignments were performed using the ClustalW algorithm in MegAlign (DNAstar, Madison Wis.). The genome sequence for NPL PEDV 2013 P10.1 was deposited in GenBank under the accession number KM052365.1.

### Chemical Inactivation

Inactivation by BEI (i.e., binary ethylenimine) is performed by mixing the viral suspension with 0.095 M BEA (2-bromo-ethylamine in 0.26 N NaOH) to a final BEI concentration of 5 mM. The virus-BEI mixture is mixed by constant stirring for a minimum of 24 hours at 36°±3° C. 2.0 M sodium thiosulfate is added to a final concentration of 30 mM to neutralize residual BEI. Mixing is continued for an additional two hours at 36°±3° C. The inactivated virus mixture is tested for residual live virus by assaying for growth on a suitable cell line. This chemical inactivation method produces enumerable structural changes, including for example, formation of new chemical bonds via chemical crosslinking and irreversible chemical alteration of the nucleic acids and protein coat (Uittenbogaard, 2011, Journal of Biological Chemistry, 286(42): pp 36198-36214; Gard, Bull. Wld Hlth Org., 1957, 17, 979-989).

### Assessment of Immunogenicity in Swine

Swine vaccination studies were performed at Newport Laboratories under biosafety level 1. Pigs approximately four weeks of age were obtained from a commercial high-health herd. Prior to study commencement pigs were verified as serologically negative to PEDV by FFN and were also negative for PEDV shedding by rt-RT-PCR on fecal swabs. Pigs were divided into eight vaccination groups of 5-9 pigs and a non-vaccinated control group of five pigs and in a single room. Pigs were allowed one week to acclimate prior to study commencement. Groups 1-3 were vaccinated intramuscularly (IM) in the neck with 2 mL of 8.0, 7.0 or 6.0 log₁₀ TCID₅₀/mL, respectively, of inactivated virus. Groups 5-7 were vaccinated IM in the neck with 2 mL of 8.0, 7.0 or 6.0 log₁₀ TCID₅₀/mL, respectively, of inactivated virus treated with Triton X-100 (added to 0.1% and incubated at room temperature 30 minutes) (Sigma). Groups 4 and 8 were vaccinated in the perineum with 8.0 log₁₀ TCID₅₀/mL of inactivated virus and inactivated virus treated with Triton X-100, respectively. All vaccines were formulated to contain 67% TS6, a proprietary oil in water adjuvant. Pigs were vaccinated on days 0 and 21. Serum was collected on days 0, 21 and 35.

### Serology

The fluorescent focus neutralization assay (FFN) was performed at South Dakota State University using a National Veterinary Services Laboratory (NVSL) reference isolate, USA/Colorado/2013 (CO/13). Briefly, test and control serum samples were heat inactivated at 56° C. for 30 minutes, then serially diluted in serum-free MEM containing 1.0 μg/ml TPCK treated trypsin in 96-well plates with a final volume of 100 μl/well. Next, 100 μl of PEDV stock diluted to 100-200 fluorescent focus units (FFU)/100 μl was added to each well and plates were incubated at 37° C. for 1 h. Plates containing confluent 3 day old monolayers of Vero-76 cells were washed 3 times with serum-free MEM prior to transfer of the serum/virus mixtures to corresponding wells of these plates. After 1 h incubation at 37° C., the serum/virus mixture was removed, monolayers washed once with serum-free MEM and 150 μl/well replacement media (MEM with 1.0 μg/ml TPCK treated trypsin) was added to each well. Plates were incubated 24 h at 37° C., then monolayers fixed for 15 min with 80% acetone in water, dried and stained with fluorescein conjugated PEDV anti-NP monoclonal antibody SD6-29. Titers were reported as the reciprocal of the greatest serum dilution resulting in a 90% or greater reduction in FFU relative to virus control well.

Enzyme-linked immunosorbent assay (ELISA) was performed at the University of Minnesota. The assay utilizes a recombinant PEDV nucleocapsid antigen and samples with a value greater than 0.5 are considered positive.

### Statistical Analysis

The Student's t-test was used to determine statistical significance of FFN titers and ELISA results using a probability value of 0.05 to indicate significance using the JMP software program (SAS, Cary, N.C.).

### Virus Isolation

The rt-RT-PCR (i.e., real time quantitative reverse transcriptase polymerase chain reaction) value of the PEDV positive intestinal homogenate was 21.4. After initial isolation attempts on Vero and Vero 76 cell lines, samples were passed on Vero cells and the amount of PEDV in the sample was quantified using rt-RT-PCR. Cytopathic effects (CPE) were evident after two passages and presence of PEDV was confirmed by rt-RT-PCR and IFA. The Ct values for passages x+1 through x+5 ranged from 17.8-23.5. Cultures were scaled to a T25 Vero flask for x+6 (17.97 CT, 4.4 log₁₀ TCID₅₀/mL). Cell cultures were adapted to M145 cells at x+7 (18.55 CT, 4.4 log₁₀ TCID₅₀/mL) and x+8 (23.31 CT and 5.2 TCID₅₀/mL) due to their USDA-licensed status for autogenous vaccine production. After two passages in M145 25 cm² flasks, the culture was scaled up to 1700 cm² roller bottles of M145 cells. This passage, X+9, had a Ct=21.2 and a titer of 6.6 log₁₀ TCID₅₀/mL as determined by IFA. The isolated PEDV was designated NPL PEDV 2013 P10.1.

### Genetic Analysis

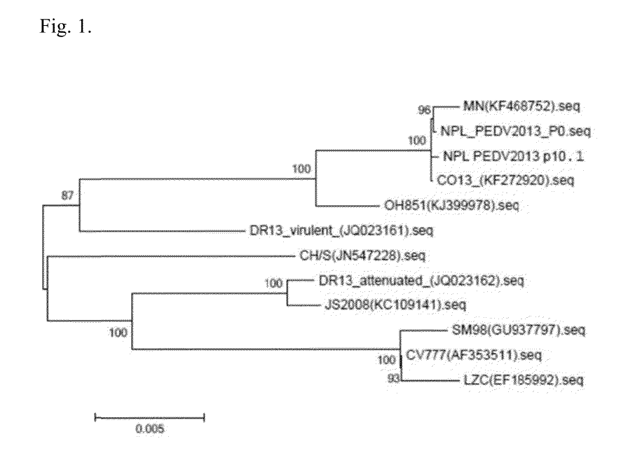

The complete genome of NPL PEDV 2013 P10.1 (SEQ ID NO. 1) was compared to the sequence derived from the original clinical sample (KJ778615) and various reference strains. The reference strains included: CV777 (EF353511) from Belgium; DR13 attenuated (JQ023162), DR13 virulent (JQ023161), and SM98 (GU937797) from South Korea; LZC (EF185992), JS2008 (KC109141), and CHS (JN547228) from China; CO13 (KF272920), MN (KF468752), and a variant strain OH851 (KJ399978) (28) from the United States. Phylogenetic analysis of complete genome sequences showed >99% identity to U.S. PED virus CO/13 (KF272920) and the original intestinal sample (KJ778615). The Minnesota isolate (KF468752) and an isolate from Ohio (KJ408801) were also closely related to the NPL PEDV2013 strain (FIG. 1). The ORFlab, spike (S), ORF3, envelope (E), membrane (M), and nucleocapsid (NP) genes of eleven PED reference viruses were aligned and the percent nucleotide identity to NPL PEDV2013 P10.1 (SEQ ID NO. 1) was determined. See Table 2.

TABLE 2

| Virus | | | | | | |
|---|---|---|---|---|---|---|
| (accession number) | ORF 1ab | S | ORF3 | E | M | NP |
| CHS (JN47228) | 98.0 | 93.8 | 98.2 | 96.5 | 98.1 | 96.8 |
| CO13 (KF272920) | 100.0 | 99.9 | 99.9 | 100.0 | 100.0 | 100.0 |
| CV777 (AF353511) | 97.3 | 94.0 | 96.9 | 97.0 | 98.2 | 96.0 |
| DR13 Attenuated | 97.8 | 93.6 | 93.1 | 96.7 | 97.9 | 96.8 |
| (DQ462404) | | | | | | |
| DR13 Virulent | 98.2 | 95.0 | 98.5 | 98.3 | 98.4 | 97.4 |
| (JQ023161) | | | | | | |
| JS2008 (KC109141) | 98.0 | 94.2 | 93.1 | 96.1 | 97.8 | 96.8 |
| LZC (EF185992) | 97.2 | 93.5 | 95.6 | 96.1 | 97.2 | 95.8 |
| MN (KJ468752) | 99.8 | 99.7 | 100.0 | 100.0 | 100.0 | 100.0 |
| OH851 (KJ399978) | 99.5 | 96.9 | 100.0 | 100.0 | 99.9 | 99.8 |
| SM98 (GU937797) | 97.2 | 93.7 | 96.8 | 96.1 | 98.1 | 95.9 |
| NPL PEDV2013 p0 | 100.0 | 99.8 | 100.0 | 100.0 | 100.0 | 100.0 |
| (KJ778615) | | | | | | |

ORF3 showed the greatest divergence, with 93.1-100% nucleotide identity. The S gene was the next most divergent, with 93.5-99.9% nucleotide identity. Amongst the US strains, ORF3, E, M, and NP were highly conserved with greater than 99.8% nucleotide identity. The S gene showed the greatest variability amongst US strains, with OH851 having 96.9% identity to NPL PEDV 2013 P10.1.

### Pig Vaccination

All pigs in the study were confirmed seronegative for PEDV antibodies at day 0 by IFA and FFN (data not shown). A FFN titer <20 was considered negative. All vaccine groups had positive geometric mean titers (GMT) by the FFN. See Table 3.

TABLE 3

| | | | FFN | |
|---|---|---|---|---|
| Group | Vaccine* | Pigs | Titer | P < 0.05† |
| 1 | 8.0 IM | 8 | 160 | A |
| 2 | 7.0 IM | 5 | 46 | B, C |
| 3 | 6.0 IM | 5 | 35 | C, D |
| 4 | 8.0 P | 9 | 254 | A |
| 5 | 8.0 IM + triton | 9 | 127 | A, B |
| 6 | 7.0 IM + triton | 5 | 92 | A, B, C |
| 7 | 6.0 IM + triton | 5 | 35 | C, D |
| 8 | 8.0 P + triton | 9 | 187 | A |
| 9 | Negative Control | 5 | 10 | D |

*PEDV titer in vaccine prior to inactivation (log₁₀ TCID₅₀/mL) and route of administration (IM, intramuscular; P, perineum).
†Groups not labeled with the same letter are significantly different from the other groups.

Group 4, which received 8.0 log₁₀ TCID₅₀/mL of inactivated virus to the perineum, had the highest FFN titer with a GMT of 254, followed by group 8 (8.0 log₁₀ TCID₅₀/mL of inactivated Triton X-100 treated virus to the perineum) with a GMT of 187. There was no statistical difference between vaccination IM to the neck or perineum for the 8.0 log₁₀ TCID₅₀/mL formulation groups (groups 1, 4, 5, 8). Group 6, which was vaccinated with 7.0 log₁₀ TCID₅₀/mL of inactivated virus treated with Triton X-100, had a GMT of 92 and was statistically similar to the 8.0 log₁₀ TCID₅₀/mL vaccine groups. Group 2, which was vaccinated with 7.0 log₁₀ TCID₅₀/mL of inactivated virus, had a GMT of only 46 which, however, was significantly greater than the negative control, group 5. The control group remained negative. The ELISA results showed only positive results in the 8.0 log₁₀ TCID₅₀/mL of inactivated virus to the perineum and IM (groups 1 and 4, Table 4).

TABLE 4

| Group | Vaccine* | Pigs | ELISA | P < 0.05† |
|---|---|---|---|---|
| 1 | 8.0 IM | 8 | 1.061 | A |
| 2 | 7.0 IM | 5 | 0.29 | B |
| 3 | 6.0 IM | 5 | <0.5 | B, C, |
| 4 | 8.0 P | 9 | 1.037 | A |
| 5 | 8.0 IM + triton | 9 | <0.5 | C |
| 6 | 7.0 IM + triton | 5 | <0.5 | B, C |
| 7 | 6.0 IM + triton | 5 | <0.5 | B, C |
| 8 | 8.0 P + triton | 9 | 0.135 | B, C |
| 9 | Negative Control | 5 | <0.5 | B, C |

*PEDV titer in vaccine prior to inactivation (log₁₀ TCID₅₀/mL) and route of administration (IM, intramuscular; P, perineum).
†Groups not labeled with the same letter are significantly different from the other groups.

Only one of the 9 animals in group 8, the 8.0 log₁₀ TCID₅₀/mL of inactivated Triton X-100 treated virus to the perineum, showed positive ELISA results.

The severity of disease caused by an outbreak of PEDV makes it imperative that an efficacious vaccine be developed. Due to the difficulties of in vitro cultivation and high virus transmissibility leading to biosecurity concerns, limited research has been performed in pigs in the U.S. With a four percent success rate for virus isolation being reported, the development of diagnostic tests and research of U.S. field strains has been hampered (6). After successfully isolating and passaging a U.S. PEDV isolate, growth was maintained on M145 cells between 5.0-6.6 TCID₅₀/mL.

The genetic characterization of NPL PEDV2013 P10.1 (SEQ ID NO. 1) found that it is 99% identical to the strains circulating in Asia in the early 2010s. Its high genetic homology to the other circulating strains in the U.S. makes it a suitable candidate for investigation of U.S. PEDV inactivated vaccine immunogenicity in pigs. While there is data published regarding the efficacy of attenuated MLVs in Asia, there is limited published data on the immunogenicity of inactivated or killed antigen PEDV vaccines.

Vaccine groups in this study were designed to look at the effects of virus titer, site of administration and detergent treatment of antigen on immunogenicity in pigs. A dose response was observed by FFN for vaccines containing different virus titers, with 8.0 log₁₀ TCID₅₀/mL groups all being significantly greater than 6.0 log₁₀ TCID₅₀/mL groups. Vaccines were administered IM or in the perineum to determine if the site of administration would affect overall immunogenic response. There was no significant difference between the two sites of administration. Likewise, there was no significant difference between vaccines formulated with triton X-100 treated antigen by FFN. A challenge model is needed to correlate FFN and/or ELISA titers to protection.

Though the vaccine in this trial was able to generate an antibody response, as indicated by FFN and ELISA assays, a protective titer is unknown. Previous work with attenuated virus used to vaccinate sows showed an immune response by ELISA in serum and colostrum, but could not draw a specific correlation to the level of mucosal immunity needed to confer protection (29). Another study showed antibody was detected in serum from piglets and colostrum from pregnant sows after being inoculated with attenuated PEDV, though finding a specific protective antibody titer of the colostrum was complicated due to varying factors including litter size, colostrum uptake per piglet, antibody concentration, and quality of colostrum (20).

Surprisingly, with the exception of one pig, negative ELISA results were obtained from pigs vaccinated with triton treated virus formulated at 8.0 log₁₀ TCID₅₀/mL despite FFN GMT's of 127 and 187. It seems likely that triton treatment of the antigen altered the antigenicity or immunogenicity of the NP, leading to negative ELISA results, while other immunogens detected in the FFN assay remained intact.

While this study focused on the humoral immune response in sera from vaccinated pigs, the post-vaccination immune response in sows and antibody titers in colostrum should be studied as the optimal vaccination regimen would utilize maternal antibodies to protect pigs when they are most susceptible to PEDV. Additionally, inactivated vaccines may prove efficacious when used as a booster in conjunction with live exposure or following MLV. PEDV continues to be a source of economic loss and has had a profound impact on the swine market in the U.S. This study demonstrates that inactivated PEDV vaccines are immunogenic in pigs.

### REFERENCES

1. Pensaert MB, de Bouck P. 1978. A new coronavirus-like particle associated with diarrhea in swine. Arch. Virol. 58:243-247. Doi:10.1007/BF01317606

2. Horvath I, Mocsari E. 1981. Ultrastructural changes in the small intestinal epithelium of suckling pigs affected with a transmissible gastroenteritis (TGE)-like disease. Arch, Virol. 68, 103-113.

3. Kweon C H, Kwon B J, Jung T S, et al. 1993. Isolation of porcine epidemic diarrhea virus (PEDV) in Korea. Korean J Vet Res 33:249-254.

4. Sueyoshi M, Tsuda T, Yamazaki K, Yoshida K, Nakaz va M, Sato K, Minimi T, Iwashita K, Watanabe M, Suzuki Y, Mori M. 1995. An Immunohistochemical Investigation of Porcine Epidemic Diarrhoea. J. Comp. Path Vol 113, 59-67

5. Arriba M L, Carvajal A, Pozo J, Rubio P. 2002. Mucosal and systemic isotype-specific antibody responses and protection in conventional pigs exposed to virulent or attenuated porcine epidemic diarrhoea virus. Veterinary Immunology and Immunopathology. 85(2002)85-97

6. Chen Q, Li G, Stasko J, Thomas J T, Stensland W R, Pillatzki A E, Gauger P C, Schwartz K J, Madson D, Yoon K J, Stevenson G W, Burrough E R, Harmon K M, Main R G, Zhang J. 2013. Isolation and Characterization of Porcine Epidemic Diarrhea Viruses Associated with the 2013 Disease Outbreak among Swine in the United. States. J. Clin, Microbiol, Jan 2014. 52:234-243

7. Huang Y W, Dickerman A W, Pineyro P, Li L, Fang L, Kiehne R, Opriessnig T, Meng X J, 2013. Origin, Evolution, and Genotyping of Emergent Porcine Epidemic Diarrhea Virus Strains in the United States. mBio vol 4 no. 5 e00737-13. Doi:10.1128/mBio.00737-13 15 Oct. 2013.

8. Shibata I, Tsuda T, Mori M, Ono M, Sueyoshi M, Uruno K. 2000. Isolation of porcine epidemic diarrhea virus in porcine cell cultures and experimental infection of pigs of different ages. Vet. Microbiol. 72 (2000) 173-182.

9. Park J E, Cruz D J M, Shin H J. 2011. Receptor-bound porcine epidemic diarrhea virus spike protein cleaved by trypsin induces membrane fusion. Arch Virol. 156:1749-1756

10. Yang X, Huo J Y, Chen L, Zheng F M, Chang H T, Zhao J, Wang X W, Wang C Q. 2012. Genetic variation analysis of reemerging porcine epidemic diarrhea virus prevailing in central China from 2010 to 2011, Virus Genes. 46:337-344

11. Li C, Li Z, Zou Y, Wicht O, van Kuppeveld F J M, et al. 2013. Manipulation of the Porcine Epidemic Diarrhea Virus Genome using targeted RNA Recombination. PloS ONE 8(8): e69997

12. Song D S, Oh J S, Kang B K, Yang J S, Moon H J, Yoo H S, king Y S, Park B K. 2007. Oral efficacy of Vero cell attenuated porcine epidemic diarrhea virus DR13 strain. Research in Vet Science 82: 134-140

13. Sato T, Takevama N, Katsumata A, Tuchiya K, Kodama T, Kusanagi K. 2011. Mutations in the spike gene of porcine epidemic diarrhea virus associated with growth adaptation in vitro and attenuation of virulence in vivo. Virus Genes 43: 72-78

14. Bosch B J, van der Zee R, de Haan C A, Rottier P J. 2003. The coronavirus spike protein is a class I virus fusion protein: structural and functional characterization of the fusion core complex. J. Virol. 77:8801-8811.

15. Sun D, Feng L, Shi H. Chen J, Cui X, Chen H, Liu S, Tong Y, Wang Y, Tong G. 2008. Identification of two novel B cell epitopes on porcine epidemic diarrhea virus spike protein. Vet. Microbiol. 131:73-811

16. Cruz D J M, Kim C H, Shin H J. 2008. The GPRLQPY motif located at the carboxy-terminal of the spike protein induces antibodies that neutralize Porcine epidemic diarrhea virus. Virus Research 132: 192-196

17. Song D, Park B. 2012. Porcine epidemic diarrhoea virus: a comprehensive review of molecular epidemiology, diagnosis, and vaccines. Virus Genes 44:167-175

18. Meng F, Ren Y, Suo S. Sun X, Li X, Li P, Yang W, Li G, Li L, Sxhwegmann-Wessels C, Herrier G, Ren X. 2013. Evaluation on the Efficacy of immunogenicity of Recombinant DNA Plasmids Expressing Spike Genes from Porcine Transmissible Gastroenteritis Virus and Porcine Epidemic Diarrhea Virus, PLOS ONE 8(3): e57468

19. Shirato K, Matsuyama 5, Ujike M, Taguchi F. 2011. Role of Proteases in the Release of Porcine Epidemic Diarrhea Virus from Infected Cells. Journal of Virology, 7872-7880.

20. Song D S, Yang J S, Oh J S, Han J H, Park B K. 2003. Differentiation of a Vero cell adapted porcine epidemic diarrhea virus from Korean field strains by restriction fragment length polymorphism analysis of ORF3. Vaccine 21:1833-1842

21. Song D S, Oh J S, Kang B K, Yang J S, Song J Y, Moon H, Kim T Y, Yoo H S, Jang Y S, Park B K. 2005. Fecal shedding of a highly cell-culture-adapted porcine epidemic diarrhea virus after oral inoculation in pigs. J Swine Health Prod. 13 (5): 269-272.

22. Chen J, Wang C, Shi H, Qiu H, Liu 5, Chen X, Zhang Z, Feng L. 2010. Molecular epidemiology of porcine epidemic diarrhea virus in China. Arch. Virol. 155:1471.-1476

23. Chen J F, Sun D B, Wang C B, Shi H Y, Cui X C, Liu S W, Qiu H J, Feng L. 2008. Molecular characterization and phylogenetic analysis of membrane protein genes of porcine epidemic diarrhea virus isolates in China. Virus Genes. 36:355-364

24. Sun R Q, Cai R J, Chen Y Q, Liang P S, Chen D K, Song C X. 2012. Outbreak of Porcine Epidemic Diarrhea in Suckling Piglets, China. Etnerg Infect Dis 18 (1): 161-163

25. Tian Y, Yu Z, Cheng K, Liu Y, Huang J, Xin Y, Li Y, Fan S, Wang T, Huang G, Feng N, Yang Z, Yang S, Gao Y, Xia X. 2013. Molecular Characterization and Phylogenetic Analysis of New Variants of the Porcine Epidemic Diarrhea Virus in Gansu, China in 2012. Viruses 2013, 5, 1991-2004

26. Kim H S, Kwang J, Yoon I J, Joo H S, Frey M L. 1993. Enhanced replication of porcine reproductive and respiratory syndrome (PRRS) virus in a homogenous subpopulation of MA-104 cell line. Arch. Virol. 133(2-4):477-83.

27. Anbalagan S. Cooper E, Kiumper P. Simonson R, Hause B. Whole genome analysis of epizootic haemorrhagic disease virus identified limited genorne constellations and preferential reassortment. Journal of General Virology,2014, 95:434-441

28. Wang L, Byrum B, Zhang Y. New variant of porcine epidemic diarrhea virus, United States, 2014 [letter]. Emerg Infect Dis. 2014 May, DOI: 10.3201/eid2005.140195

29. Kweon C H, Kwon B J, Lee J G, Kwon G O, Kang Y B. 1999. Derivation of attenuated porcine epidemic diarrhea virus (PEDV) as vaccine candidate. Vaccine. 17:2546-2553.

1. A composition or vaccine comprising a virus encoded by SEQ ID NO. 1.

2. The composition or vaccine of claim 1, wherein the virus encoded by SEQ ID NO. 1 is inactivated.

3. The composition or vaccine of claim 1, wherein the composition or vaccine further comprises one or more pharmaceutically or veterinarily acceptable carriers, adjuvants, vehicles or excipients.

4. A composition or vaccine comprising a protein encoded by SEQ ID NO. 10.

5. The composition or vaccine of claim 4, wherein the protein encoded by SEQ ID NO. 10 is inactivated.

6. The composition or vaccine of claim 4, wherein the composition or vaccine further comprises one or more pharmaceutically or veterinarily acceptable carriers, adjuvants, vehicles or excipients.

7. A method of vaccinating a host susceptible to porcine epidemic diarrhea virus comprising at least one administration of a composition or vaccine according to claim 3.

8. A method of vaccinating a host susceptible to porcine epidemic diarrhea virus comprising at least one administration of a composition or vaccine according to claim 6.
